(19) United States
(12) Patent Application Publication
(10) Pub. No.: US 2024/0173471 A1
(43) Pub. Date: May 30, 2024

(54) MEDICAMENT INJECTION PEN WITH AUTOMATIC DOSING

(71) Applicant: Dexcom, Inc., San Diego, CA (US)

(72) Inventor: Edward J. Sweeney, San Diego, CA (US)

(21) Appl. No.: 18/502,453

(22) Filed: Nov. 6, 2023

Related U.S. Application Data
(60) Provisional application No. 63/428,855, filed on Nov. 30, 2022.

(51) Int. Cl. A61M 5/142

(52) U.S. Cl. CPC A61M 5/14244

(57) ABSTRACT

Automatic medicament dosing is described. A dose of a medicament to administer to a user via a medicament injection pen is calculated based on analyte monitoring data. A user input to initiate an injection of the medicament is received, and the medicament injection pen is operated to administer the calculated dose to the user in response to receiving the user input. In one or more implementations, operating the medicament pump to administer the calculated dose to the user includes powering a motorized pump of the medicament injection pen to flow the calculated dose from a medicament reservoir through a needle. In one or more implementations, the medicament pen is communicatively coupled to a computing device, and operating the medicament pump to administer the calculated dose to the user is further in response to the medicament injection pen receiving instructions to administer the calculated dose from the computing device.

### BACKGROUND TO RELATED APPLICATION

This application claims the benefit of U.S. Provisional Application 63/428,855, filed Nov. 30, 2022 entitled “Medicament Injection Pen with Automatic Dosing”, the contents of which are incorporated herein by reference.

### BACKGROUND

Diabetes is a metabolic condition affecting hundreds of millions of people. For these people, monitoring blood glucose levels and regulating those levels to be within an acceptable range is important not only to mitigate long-term issues such as heart disease and vision loss, but also to avoid the effects of hyperglycemia and hypoglycemia. Maintaining blood glucose levels within an acceptable range can be challenging, as this level is almost constantly changing over time and in response to everyday events and activities, such as eating, exercising, sleeping, and stress.

### SUMMARY

To address these problems, a medicament injection pen with automatic dosing is leveraged. A dose of a medicament to administer to a user via the medicament injection pen is calculated based on analyte monitoring data. A user input to initiate an injection of the medicament is received, and the medicament injection pen is operated to administer the calculated dose to the user in response to receiving the user input. In one or more implementations, operating the medicament pen to administer the calculated dose to the user includes powering a motorized pump of the medicament injection pen to flow the calculated dose from a medicament reservoir through a needle. In one or more implementations, the medicament pen is communicatively coupled to a computing device, and operating the medicament pump to administer the calculated dose to the user is further in response to the medicament injection pen receiving instructions to administer the calculated dose from the computing device.

In a first aspect, a method is presented in which a dose is calculated of a medicament to administer to a user via a medicament injection pen based on analyte monitoring data. User input is received to initiate an injection of the medicament. The medicament injection pen is operated to administer the calculated dose to the user in response to receiving the user input to initiate the injection.

In an embodiment of the first aspect, the operating of the medicament injection pen to administer the calculated dose includes powering a motorized pump of the medicament injection pen to flow the calculated dose from a medicament reservoir of the medicament injection pen through a needle of the medicament injection pen.

In another embodiment of the first aspect, the method further includes estimating an amount of active medicament present in the user based on a dosage log of previously injected doses of the medicament, and wherein the calculating the dose of the medicament to administer to the user via the medicament injection pen is further based on the amount of active medicament present in the user.

In another embodiment of the first aspect, the method further includes determining a blood glucose level of the user based on the analyte monitoring data, the analyte monitoring data received from an analyte monitoring device worn by the user, and wherein the calculating the dose of the medicament to administer to the user via the medicament injection pen based on the analyte monitoring data is in response to the blood glucose level of the user being greater than a threshold.

In another embodiment of the first aspect, the method further includes outputting a notification regarding the calculated dose prior to receiving the user input to initiate the injection.

In another embodiment of the first aspect, the outputting of the notification includes outputting the notification via a user interface of the medicament injection pen, and wherein the receiving the user input to initiate the injection comprises receiving the user input via the user interface or an electronic button of the medicament injection pen.

In another embodiment of the first aspect, the outputting of the notification includes outputting the notification via a user interface of a computing device that is communicatively coupled to the medicament injection pen.

In another embodiment of the first aspect, the notification includes a first notification indicating the calculated dose of the medicament, and wherein the receiving the user input to initiate the injection comprises receiving a first user input verifying the calculated dose of the medicament after outputting the first notification.

In another embodiment of the first aspect, the method further includes outputting, in response to receiving the first user input, a second notification including instructions to position the medicament injection pen to inject the calculated dose of the medicament into the user, and wherein the receiving the user input to initiate the injection further comprises receiving a second user input confirming positioning of the medicament injection pen after outputting the second notification.

In another embodiment of the first aspect, the medicament injection pen is communicatively coupled to a computing device, and the operating the medicament injection pen to administer the calculated dose to the user is further in response to the medicament injection pen receiving, from the computing device, instructions to administer the calculated dose.

In another embodiment of the first aspect, the receiving of the user input to initiate the injection of the medicament includes receiving the user input via a user interface of the computing device.

In another embodiment of the first aspect, the calculating of the dose of the medicament to administer to the user via the medicament injection pen based on the analyte monitoring data is performed by the computing device in response to a communicative coupling between the computing device and a continuous medicament delivery pump being disconnected.

In a second aspect, a system is presented that includes an analyte monitoring device, a medicament injection pen, and at least a memory and a processor. The analyte monitoring device is configured to obtain analyte measurements of a user. The medicament injection pen is configured to administer a medicament to the user. The memory and processor are configured to perform operations that include: calculating, based on the analyte measurements, a blood glucose level of the user; calculating an amount of the medicament to administer to the user in response to the blood glucose level of the user exceeding a threshold; and providing instructions causing the medicament injection pen to deliver the amount of the medicament to the user in response to receiving an indication that a needle of the medicament injection pen is inserted into the user.

In an embodiment of the second aspect, the amount of the medicament is determined based at least in part on the analyte measurements of the user obtained from the analyte monitoring device.

In another embodiment of the second aspect, the instructions cause the medicament injection pen to deliver the amount of the medicament to the user without receiving user input setting the amount.

In another embodiment of the second aspect, the medicament comprises insulin, and wherein the medicament injection pen comprises a motorized pump.

In another embodiment of the second aspect, the medicament injection pen has a user interface for receiving user input causing the medicament injection pen to initiate delivery of the amount of the medicament to the user.

In another embodiment of the second aspect, the medicament injection pen includes a user interface for establishing wireless communication with an external device.

In another embodiment of the second aspect, the external devices is the analyte monitoring system.

In another embodiment of the second aspect, the external device includes a computing device in which the at least memory and the processor are located.

In another embodiment of the second aspect, the medicament injection pen includes a user interface that selectively activates a retraction mechanism to extend and retract the needle from a cap.

In another embodiment of the second aspect, the memory and the processor are included in the medicament injection pen.

In another embodiment of the second aspect, the system further includes a computing device in which the memory and the processor are located, the medicament injection pen including a communication module for receiving the instructions from the computing device.

In another embodiment of the second aspect, the computing device receives the analyte measurements from the analyte monitoring device over a wireless connection.

In another embodiment of the second aspect, the motorized pump includes a stepper motor in which each rotation is divided into a pre-defined number of steps.

In another embodiment of the second aspect, each of the steps of the stepper motor causes a known quantity of the medicament to be delivered via the needle.

In another embodiment of the second aspect, the memory and the processor calculate the number of steps to rotate the stepper motor to extract the amount of medicament to be delivered via the needle.

In another embodiment of the second aspect, the medicament injection pen includes a medicament reservoir in which the medicament is housed, wherein the medicament injection pen is configured to identify a type of medicament in the medicament reservoir based on an indicator in the medicament reservoir.

In another embodiment of the second aspect, the medicament injection pen includes a display for displaying analyte measurements.

In another embodiment of the second aspect, the medicament injection pen includes a medicament sensor for measuring a systemic level of the medicament in the user.

In another embodiment of the second aspect, the medicament injection pen includes a safety module for preventing medicament delivery based on medicament measurements from the medicament sensor.

In another embodiment of the second aspect, the amount of the medicament to administer is a bolus dose and calculating the amount includes dividing the bolus dose into a plurality of smaller doses that are to be administered at intervals determined by the memory and the processor.

In another embodiment of the second aspect, determining to divide the bolus does into a plurality of smaller doses is based on receipt of user input.

In a third aspect, a method is presented that includes calculating a bolus amount of medicament to administer to a user via a medicament injection pen in response to a blood glucose level of the user exceeding a threshold. An indication that a needle of the medicament injection pen is inserted into the user is received. A motorized pump of the medicament injection pen is operated to deliver the bolus amount of the medicament to the user via at least one injection in response to receiving the indication that the needle of the medicament injection pen is inserted into the user.

In an embodiment of the third aspect, the method further includes obtaining glucose measurements of the user from a glucose monitoring device worn by the user, and the calculating of the bolus amount of the medicament to administer to the user in response to the blood glucose level of the user exceeding a threshold includes calculating the bolus amount based at least in part on the glucose measurements.

In another embodiment of the third aspect, the method further includes determining an active amount of the medicament in the user based at least in part on a dosage log of medicament delivery to the user, and wherein the calculating the bolus amount of the medicament to administer to the user in response to the blood glucose level of the user exceeding a threshold comprises calculating the bolus amount further based on the active amount of the medicament.

In another embodiment of the third aspect, the method further includes outputting at least one notification regarding administering the calculated bolus amount of the medicament to the user via a user interface, and the receiving of the indication that the needle of the medicament injection pen is inserted into the user includes receiving the indication via the user interface after outputting the notification.

In another embodiment of the third aspect, the bolus amount of the medicament is determined based at least in part on the analyte measurements of the user obtained from an analyte monitoring device.

In another embodiment of the third aspect, the bolus amount of the medicament is delivered to the user without receiving user input setting the amount. In another embodiment of the third aspect, the medicament injection pen has a user interface for receiving user input causing the motorized pump to initiate delivery of the amount of the medicament to the user.

In another embodiment of the third aspect, the method further includes establishing wireless communication between the medicament injection pen and an external device.

In another embodiment of the third aspect, the external devices is a glucose monitoring system that is configured to obtain the blood glucose level of the user.

In another embodiment of the third aspect, the external device includes a computing device the computing device performing the calculating and communicating the calculated bolus amount to the medicament injection pen.

In another embodiment of the third aspect, the method further includes receiving user input that selectively activates a retraction mechanism in the medicament injection pen to extend and retract the needle from a cap.

In another embodiment of the third aspect, the calculating is performed by the medicament injection pen.

In another embodiment of the third aspect, the motorized pump includes a stepper motor in which each rotation is divided into a pre-defined number of steps.

In another embodiment of the third aspect, each of the steps of the stepper motor causes a known quantity of the medicament to be delivered via the needle.

In another embodiment of the third aspect, the method further includes calculating the number of steps to rotate the stepper motor to extract the amount of medicament to be delivered via the needle.

In another embodiment of the third aspect, the method further includes identifying a type of medicament in a medicament reservoir of the medicament injection pen based on an indicator in the medicament reservoir.

In another embodiment of the third aspect, the method further includes receiving medicament measurements data from a medicament sensor, the medicament measurements indicating a systemic level of the medicament in the user.

In another embodiment of the third aspect, the method further includes preventing medicament delivery based on the medicament measurements from the medicament sensor.

In another embodiment of the third aspect, calculating the bolus amount includes dividing the bolus amount into a plurality of smaller doses that are to be administered at intervals determined by the memory and the processor.

In another embodiment of the third aspect, the method further includes receiving user input that causes the bolus amount to be divided into the plurality of smaller doses.

This Summary introduces a selection of concepts in a simplified form that are further described below in the Detailed Description. As such, this Summary is not intended to identify essential features of the claimed subject matter, nor is it intended to be used as an aid in determining the scope of the claimed subject matter.

### BRIEF DESCRIPTION OF THE DRAWINGS

The detailed description is described with reference to the accompanying figures.

FIG. 1 is an illustration of an environment in an example implementation that is operable to employ automatic glycemic control.

FIG. 2 depicts an example implementation of an analyte monitoring device.

FIG. 3 depicts an example implementation of a medicament delivery system.

FIG. 4 depicts an example of a system that receives and processes sensor data from various sources to generate medicament dosing instructions.

FIG. 5 depicts an example of a user interface displaying an intervention notification.

FIG. 6 depicts an additional example of a user interface displaying an intervention notification.

FIG. 7 depicts an additional example of a user interface displaying an intervention notification.

FIG. 8 depicts an example of a first combination of devices for implementing automatic medicament dosing.

FIG. 9 depicts an example of a second combination of devices for implementing automatic medicament dosing.

FIG. 10 depicts an example of a third combination of devices for implementing automatic medicament dosing.

FIG. 11 depicts a procedure in an example implementation of automatic medicament dosing.

FIG. 12 depicts a procedure in an example implementation of automatic medicament dosing using an automatic medicament dosing injection pen.

FIG. 13 illustrates an example of a system that includes an example of a computing device that is representative of one or more computing systems and/or devices that may implement the various techniques described herein.

### DETAILED DESCRIPTION

### Overview

Individuals having diabetes (e.g., type 1 or type 2 diabetes) may utilize a medicament, such as insulin, to help regulate blood glucose levels. High blood glucose (e.g., hyperglycemia) may cause a variety of adverse health effects, such as tiredness and blurred vision. Low blood sugar (e.g., hypoglycemia) may also cause adverse health effects, such as dizziness and confusion, and can be induced by an inadvertent overdose of insulin or occur after a normal dose of insulin (or other glucose-lowering agent) is given accompanied by higher-than-average exercise or insufficient food intake.

Different types of blood glucose monitors and medicament delivery devices are available for monitoring blood glucose levels and regulating those levels, ranging from fully manual to fully automatic. In an example of a fully manual approach, a diabetic individual may use a finger prick-based blood glucose monitor to measure a current blood glucose level and manually calculate and inject the medicament (e.g., via an injection pen or syringe) based on the measured blood glucose level. However, this calculation and subsequent injection is subject to user error, which may increase a likelihood that the individual will deliver too much medicament or not enough medicament to maintain his or her blood glucose levels within a desired range. Furthermore, due to the lack of comfort and convenience, the individual may only measure his or her glucose level two to four times per day, and these measurements may be so spread apart that the individual may not identify a hyperglycemic or hypoglycemic condition in a timely manner. As a result, the individual may experience adverse health effects.

In an example of a fully automatic approach, a continuously worn pump-based delivery system may deliver the medicament around the clock, with the delivered dose adjusted based on measurements from a continuous glucose monitoring device. For example, the continuously worn pump-based delivery system and the continuous glucose monitoring device may operate in a closed-loop system to maintain the individual's blood glucose level within the desired range. As a result, an accuracy of controlling the individual's blood glucose level is increased, and the individual may experience fewer hyperglycemic and hypoglycemic events, and thus fewer adverse health effects, compared with the fully manual approach. However, the continuously worn pump-based delivery system may be expensive. As another example, the individual may tire of wearing the pump-based delivery system.

In an example of a partially manual approach, the individual may use the continuous glucose monitoring device to obtain blood glucose level measurements around the clock but may manually set and perform the medicament injection. In contrast to the around-the-clock delivery of the continuously worn pump-based system, the individual may perform the medicament injection several times a day, such as before meals or in response to the measured blood glucose level reaching an upper threshold. For example, the individual may use a bolus calculator to suggest a dose of the medicament based on measurements received from the continuous glucose monitoring device, and the individual may then manually set an injection device (e.g., the injection pen or syringe) to the suggested dose and perform the injection. However, this approach is subject to user error, as the individual may inadvertently set the delivery device to the wrong dose. As another example, the individual may have reduced control over the amount of medicament in the dose compared to the pump-based system due to, for example, a precision of syringe markings (as well as the individual's accuracy of filling the syringe) or dial settings of the injection pen. Thus, even when the dose is automatically calculated, an individual may still struggle to provide accurate medicament injections using currently available injection devices.

Each individual may utilize one or a combination of the above-described delivery devices. For example, an individual may occasionally take breaks from wearing the pump-based delivery system and switch to using a fully manual or partially manual injection pen. However, this individual may not be used to self-managing his or her blood glucose levels, increasing a likelihood that an incorrect dose will be delivered via the injection pen. As another example, individuals who have disabilities, such as vision impairment, may experience difficulties manually setting a dose to be injected.

To overcome these problems, a medicament injection pen with automatic dosing is described. In accordance with the described techniques, a glycemic control system is configured to receive analyte monitoring data (e.g., glucose monitoring data) from an analyte monitoring device worn by a user and calculate a dose of medicament to provide to the user based on the analyte monitoring data. The glycemic control system is further configured to provide instructions to the medicament injection pen to administer the calculated dose of the medicament to the user. The medicament injection pen automatically provides the calculated dose to the user, without the user having to set the dose.

Because the user manually inserts a needle of the medicament injection pen to administer the medicament, the medicament injection pen may receive at least one user input to initiate the injection. By way of example, the medicament injection pen may receive a first user input that confirms the calculated dose and a second user input that triggers injection of the calculated dose. However, when the glycemic control system is utilized for automatic dosing, the at least one user input may not include input to set the dose of the medicament.

In at least one implementation, the medicament injection pen operates a pump included therein to dispense the calculated dose. By way of example, the instructions to administer the calculated dose of the medicament to the user may include instructions for operating the pump. In at least one implementation, the pump is a motorized pump having a stepper motor, where each step of the stepper motor dispenses a known amount of the medicament. As such, the instructions may define a number of steps to rotate the stepper motor in order to administer the calculated dose. Furthermore, the motorized pump may have finer control of the amount of medicament administered compared with, for example, dial settings of a conventional injection pen.

In some cases, the glycemic control system calculates the dose of the medicament to provide to the user in response to a blood glucose level of the user exceeding a threshold. Thus, the dose of the medicament may be a bolus dose calculated in response to a high blood glucose excursion. In some scenarios, the bolus dose is divided between multiple injections administered at intervals determined via the glycemic control system. Dividing the bolus dose into multiple injections may result in finer blood glucose level control, such as by more gradually lowering the blood glucose level via the medicament. Some individuals may prefer the multiple injections, while other individuals may prefer a single injection. Thus, in at least one implementation, the glycemic control system decides between delivering the calculated dose as a single injection or multiple smaller injections based on user preferences received via user input. A cumulative dose of the medicament given via the multiple smaller injections may be different (e.g., smaller or larger) than a dose of a comparable single injection due to different delivery timings.

In one or more implementations, a computing device that is communicatively coupled to the medicament injection pen and the analyte monitoring device includes the glycemic control system. In this scenario, the computing device receives the analyte monitoring data from the analyte monitoring device (e.g., via a first wireless connection), calculates the dose of the medicament to provide to the user based on the analyte monitoring data, generates instructions for providing the calculated dose of the medicament via the medicament injection pen, and transmits the instructions to the medicament injection pen (e.g., via a second wireless connection). The medicament injection pen may receive and execute the instructions. In at least one other implementation, the medicament injection pen is communicatively coupled to the analyte monitoring device and includes the glycemic control system. In this scenario, the medicament injection pen receives the analyte monitoring data from the analyte monitoring device (e.g., via a wireless connection), calculates the dose of the medicament to provide to the user based on the analyte monitoring data, generates the instructions for providing the calculated dose of the medicament, and executes the instructions.

In some cases, the computing device is capable of sending and receiving communication signals with the medicament injection pen and a continuous medicament delivery pump. In some such examples where the glycemic control system is included on the computing device, the glycemic control system calculates the dose of the medicament to provide to the user via the medicament injection pen in response to the continuous medicament delivery pump not being connected and/or the medicament injection pen being connected. By way of example, the glycemic control system may use different algorithms to calculate medicament delivery amounts when the continuous medicament delivery pump is connected (and thus indicated to be in use) versus when the continuous medicament delivery pump is not connected (and thus indicated to not be in use). As an example, the continuous medicament delivery pump may periodically or continuously provide smaller, basal doses of the medicament throughout a 24-hour period, whereas the medicament delivery pen may provide larger, bolus doses of the medicament via one or more discrete injections throughout the 24-hour period. Consider, for example, that the user may take breaks from wearing the continuous medicament delivery pump, and during these breaks, the continuous medicament delivery pump may be powered off or otherwise out of communication with the computing device. During these breaks, the user may instead use the medicament injection pen to provide the medicament, and so the glycemic control system may switch to calculating doses of the medicament to deliver via discrete injections, rather than the periodic or continuous doses of the medicament provided via the continuous medicament delivery pump, in order to provide accurate blood glucose control via the medicament injection pen.

The medicament injection pen, the computing device (when connected), or a combination thereof may output notifications. By way of example, the medicament injection pen and/or the computing device may output a notification indicating that a medicament injection is recommended and the calculated dose of the medicament to be injected when the blood glucose level of the user exceeds the threshold. In some examples, additional notifications are output that indicate user instructions and/or a status of the medicament injection pen. For example, the medicament injection pen may output, via a user interface, a “dosing in progress” status while the medicament injection pen is operating the pump to dispense the calculated dose and/or a “dosing complete” status after dispensing the calculated dose.

Moreover, the medicament injection pen, the computing device (when connected), or a combination thereof may receive user input. As mentioned above, the medicament injection pen may receive at least one user input to initiate the injection. By way of example, the medicament injection pen may receive the user input directly, such as via the user interface or a button (e.g., an electrical button), or indirectly, such as via inputs to the computing device. An additional example of a user input includes a priming input configured to prime the needle of the medicament injection pen by operating the pump when the needle is not inserted in the user. Priming the needle may push air out of the needle, for example, by replacing the air with the medicament delivered by the pump.

By automatically calculating and dispensing a dose of the medicament via the medicament injection pen using the glycemic control system, the medicament is more accurately provided to the user with decreased user interaction and a decreased mental burden on the user. Furthermore, the medicament injection pen with automatic dosing described herein has increased accessibility for users with disabilities or impairments, such as vision impairments, compared with medicament injection pens that require user input to set the dosing. By more accurately controlling the amount of medicament provided to the user, adverse health effects associated with hypoglycemia and hyperglycemia may be avoided.

A technical effect of calculating a dose of a medicament to provide to a user based on analyte measurements of the user and automatically dispensing the calculated dose from a medicament injection pen by operating a pump of the medicament injection pen according to the calculated dose is that elevated blood glucose excursions in the user may be accurately mitigated with decreased adverse health effects.

In some aspects, the techniques described herein relate to a method including: calculating a dose of a medicament to administer to a user via a medicament injection pen based on analyte monitoring data; receiving a user input to initiate an injection of the medicament; and operating the medicament injection pen to administer the calculated dose to the user in response to receiving the user input to initiate the injection.

In some aspects, the techniques described herein relate to a method, wherein the operating the medicament injection pen to administer the calculated dose of the medicament to the user in response to receiving the user input to initiate the injection includes powering a motorized pump of the medicament injection pen to flow the calculated dose from a medicament reservoir of the medicament injection pen through a needle of the medicament injection pen.

In some aspects, the techniques described herein relate to a method, further including estimating an amount of active medicament present in the user based on a dosage log of previously injected doses of the medicament, and wherein the calculating the dose of the medicament to administer to the user via the medicament injection pen is further based on the amount of active medicament present in the user.

In some aspects, the techniques described herein relate to a method, further including determining a blood glucose level of the user based on the analyte monitoring data, the analyte monitoring data received from an analyte monitoring device worn by the user, and wherein the calculating the dose of the medicament to administer to the user via the medicament injection pen based on the analyte monitoring data is in response to the blood glucose level of the user being greater than a threshold.

In some aspects, the techniques described herein relate to a method, further including outputting a notification regarding the calculated dose prior to receiving the user input to initiate the injection.

In some aspects, the techniques described herein relate to a method, wherein the outputting the notification includes outputting the notification via a user interface of the medicament injection pen, and wherein the receiving the user input to initiate the injection includes receiving the user input via the user interface or an electronic button of the medicament injection pen.

In some aspects, the techniques described herein relate to a method, wherein outputting the notification includes outputting the notification via a user interface of a computing device that is communicatively coupled to the medicament injection pen.

In some aspects, the techniques described herein relate to a method, wherein the notification includes a first notification indicating the calculated dose of the medicament, and wherein the receiving the user input to initiate the injection includes receiving a first user input verifying the calculated dose of the medicament after outputting the first notification.

In some aspects, the techniques described herein relate to a method, further including outputting, in response to receiving the first user input, a second notification including instructions to position the medicament injection pen to inject the calculated dose of the medicament into the user, and wherein the receiving the user input to initiate the injection further includes receiving a second user input confirming positioning of the medicament injection pen after outputting the second notification.

In some aspects, the techniques described herein relate to a method, wherein the medicament injection pen is communicatively coupled to a computing device, and the operating the medicament injection pen to administer the calculated dose to the user is further in response to the medicament injection pen receiving, from the computing device, instructions to administer the calculated dose.

In some aspects, the techniques described herein relate to a method, wherein the receiving the user input to initiate the injection of the medicament includes receiving the user input via a user interface of the computing device.

In some aspects, the techniques described herein relate to a method, wherein the calculating the dose of the medicament to administer to the user via the medicament injection pen based on the analyte monitoring data is performed by the computing device in response to a communicative coupling between the computing device and a continuous medicament delivery pump being disconnected.

In some aspects, the techniques described herein relate to a system including: an analyte monitoring device configured to obtain analyte measurements of a user; a medicament injection pen to administer a medicament to the user; and at least a memory and a processor to perform operations including: calculating, based on the analyte measurements, a blood glucose level of the user; calculating an amount of the medicament to administer to the user in response to the blood glucose level of the user exceeding a threshold; and communicating instructions to the medicament injection pen, the instructions causing the medicament injection pen to deliver the amount of the medicament to the user in response to receiving an indication that a needle of the medicament injection pen is inserted into the user.

In some aspects, the techniques described herein relate to a system, wherein the amount of the medicament is determined based at least in part on the analyte measurements of the user obtained from the analyte monitoring device.

In some aspects, the techniques described herein relate to a system, wherein the instructions cause the medicament injection pen to deliver the amount of the medicament to the user without receiving user input setting the amount.

In some aspects, the techniques described herein relate to a system, wherein the medicament includes insulin, and wherein the medicament injection pen includes a motorized pump.

In some aspects, the techniques described herein relate to a method including: calculating a bolus amount of medicament to administer to a user via a medicament injection pen in response to a blood glucose level of the user exceeding a threshold; receiving an indication that a needle of the medicament injection pen is inserted into the user; and operating a motorized pump of the medicament injection pen to deliver the bolus amount of the medicament to the user via at least one injection in response to receiving the indication that the needle of the medicament injection pen is inserted into the user.

In some aspects, the techniques described herein relate to a method, further including obtaining glucose measurements of the user from a glucose monitoring device worn by the user, and wherein the calculating the bolus amount of the medicament to administer to the user in response to the blood glucose level of the user exceeding a threshold includes calculating the bolus amount based at least in part on the glucose measurements.

In some aspects, the techniques described herein relate to a method, further including determining an active amount of the medicament in the user based at least in part on a dosage log of medicament delivery to the user, and wherein the calculating the bolus amount of the medicament to administer to the user in response to the blood glucose level of the user exceeding a threshold includes calculating the bolus amount further based on the active amount of the medicament.

In some aspects, the techniques described herein relate to a method, further including outputting at least one notification regarding administering the calculated bolus amount of the medicament to the user via a user interface, and wherein the receiving the indication that the needle of the medicament injection pen is inserted into the user includes receiving the indication via the user interface after outputting the at least one notification.

In the following discussion, an exemplary environment is first described that may employ the techniques described herein. Examples of implementation details and procedures are then described which may be performed in the exemplary environment as well as other environments. Performance of the exemplary procedures is not limited to the exemplary environment and the exemplary environment is not limited to performance of the exemplary procedures.

### Example of an Environment

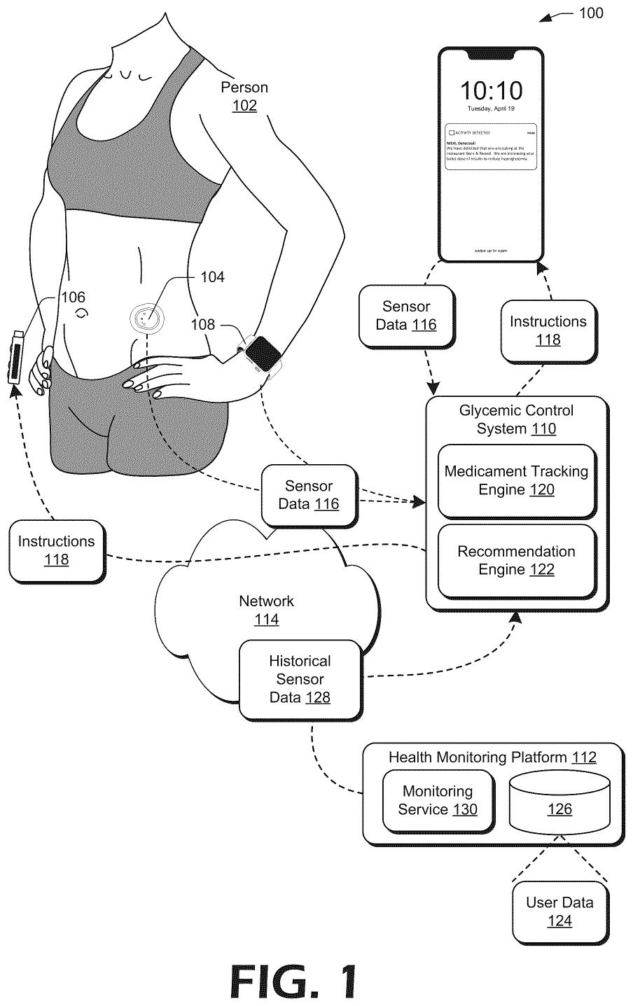

FIG. 1 is an illustration of an environment 100 in an example implementation that is operable to employ a medicament injection pen with automatic dosing. The illustrated environment 100 includes person 102, who is depicted wearing analyte monitoring device 104, and a medicament delivery system 106. The illustrated environment 100 also includes example computing devices 108, a glycemic control system 110, and a health monitoring platform 112. The analyte monitoring device 104, the medicament delivery system 106, the example computing devices 108, the glycemic control system 110, and the health monitoring platform 112, are communicably coupled, including via network 114.

The analyte monitoring device 104, the medicament delivery system 106, and one or more computing devices 108 (associated with the person 102) may be communicably coupled in various ways, such as by using one or more wireless communication protocols or techniques. By way of example, the analyte monitoring device 104, the medicament delivery system 106, and one or more computing devices 108 may communicate with one another using one or more of radio, cellular, Wi-Fi, Bluetooth® (e.g., Bluetooth® Low Energy links), near-field communication (NFC), and 5G, to name just a few.

Through such communicative couplings, the analyte monitoring device 104, the medicament delivery system 106, and one or more of the computing devices 108 are configured to provide glycemic control in a way that eliminates or reduces user decisions and interactions involved with mitigating adverse health events (e.g., glycemic events). Instead, the devices process various information and make various predictions and determinations, such as about therapies to mitigate those glycemic events, such as elevated blood glucose.

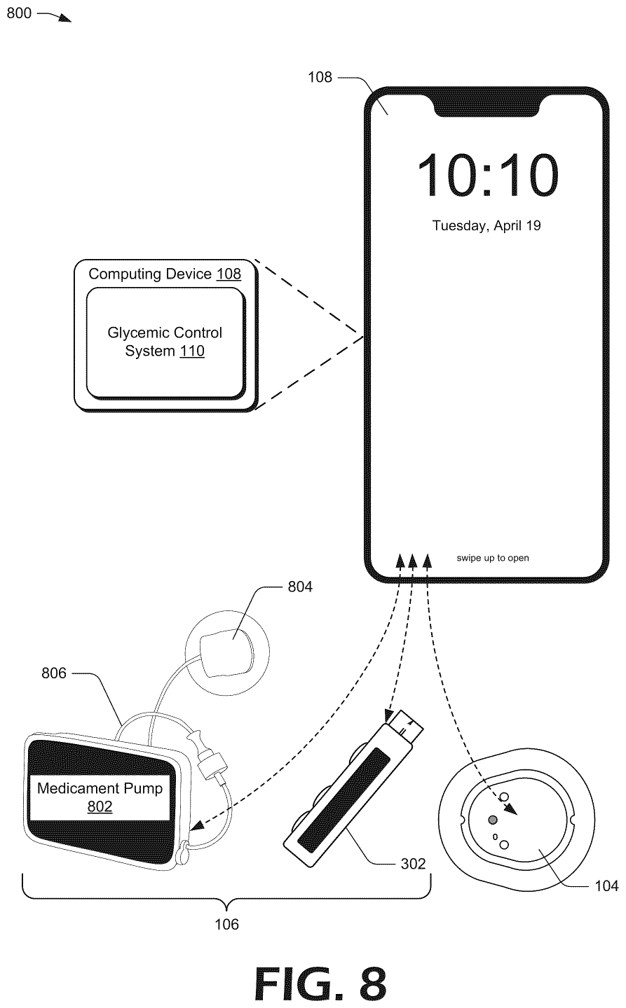

In one or more implementations, the analyte monitoring device 104, the medicament delivery system 106, and one or more of the computing devices 108 form a closed-loop system. In a closed-loop system, the devices can then provide the determined therapy without user interaction. For example, the medicament delivery system 106 controlled to administer medicament to the person 102 based on feedback from the analyte monitoring device 104. The medicament delivery system 106 may be controlled to administer basal doses of the medicament, bolus doses of the medicament, or combinations thereof. In the context of continuously worn medicament delivery pumps, a “basal dose” refers to periodic or continuous delivery of low levels of the medicament, such as throughout a 24-hour period. In the context of medicament injection pens, a “basal dose” refers to a dose of a longer-acting medicament, such as long-acting insulin, that acts throughout the 24-hour period. In contrast, a “bolus dose” refers to a dose of medicament that is given over a short, defined period of time. In one non-limiting example, a bolus dose is given to cover an expected rise in blood glucose, such as generally occurs during and/or after a meal. According to the techniques described herein, the bolus dose may be given as one or multiple injections. Furthermore, in at least one implementation, the person 102 may utilize more than one delivery device in the medicament delivery system 106, an example of which will be described in more detail with respect to FIG. 8. For example, the person 102 may alternate between a wearable medicament delivery system and a hand-held medicament delivery system during a same day or on different days.

In one or more implementations, the analyte monitoring device 104 is a wearable, such that it is worn by the person 102 while the device performs various operations. Additionally, or alternatively, the analyte monitoring device 104 performs one or more operations before or after being worn by the person 102. Broadly, the analyte monitoring device 104 is configured to provide measurements of an analyte of the person 102 (e.g., the person 102's glucose). For instance, the analyte monitoring device 104 may be configured with an analyte sensor that detects one or more signals indicative of the analyte in the person 102 and enables generation of analyte measurements or estimations (e.g., estimated glucose values). Those analyte measurements (e.g., glucose measurements) may correspond to or otherwise be packaged for communication to one or more of the computing devices 108 or the medicament delivery system 106 as analyte data, which is one example of sensor data 116.

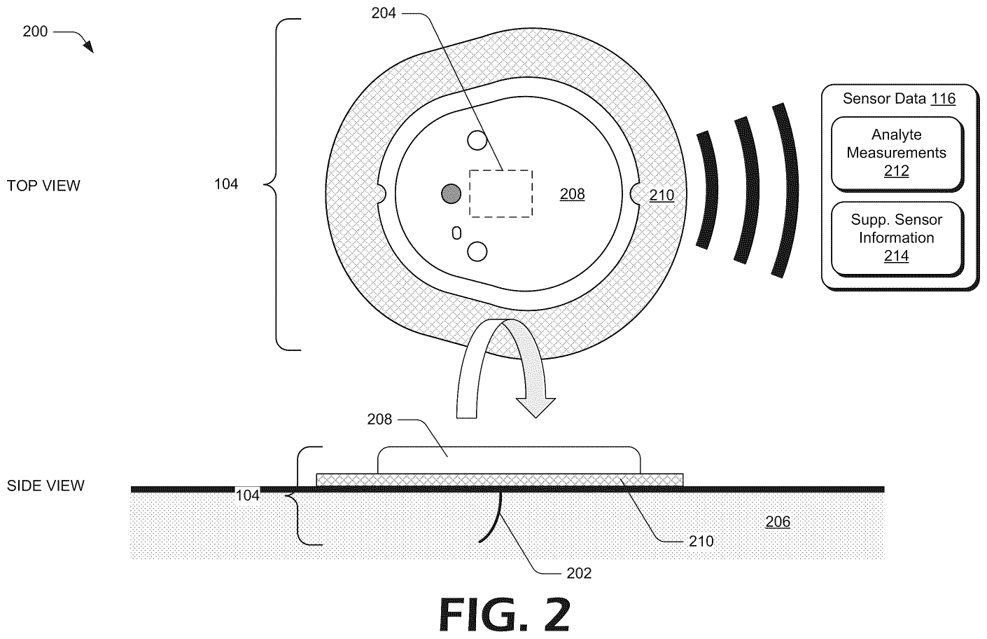

In at least one implementation, the analyte monitoring device 104 is a glucose monitoring system. As an example, the analyte monitoring device 104 may be configured as a continuous glucose monitoring (“CGM”) system (e.g., a wearable CGM system). As used herein, the term “continuous” when used in connection with analyte monitoring may refer to an ability of a device to produce measurements substantially continuously, such that the device may be configured to produce the analyte measurements at regular or irregular intervals of time (e.g., approximately every hour, approximately every 30 minutes, approximately every 5 minutes, and so forth), responsive to establishing a communicative coupling with a different device (e.g., when a computing device 108 establishes a wireless connection with the analyte monitoring device 104 to retrieve one or more of the measurements), and so forth. In other implementations, however, the glucose monitoring device may not be “continuous”, and instead provides glucose measurements when requested. This functionality along with further aspects of the configuration of the analyte monitoring device 104 are discussed in more detail in relation to FIG. 2.

In addition to producing sensor data 116 (including analyte data indicative of measurements of the analyte in the person 102), the analyte monitoring device 104 also transmits the produced sensor data 116, for example, to the computing device 108. The analyte monitoring device 104 may communicate the data in real-time (e.g., as it is produced using an analyte sensor or other sensors). Alternatively, or in addition, the analyte monitoring device 104 may communicate the data to the computing device 108 at predefined intervals of time. For example, the analyte monitoring device 104 may be configured to communicate the sensor data 116 to the computing device 108 approximately every five minutes. Certainly, an interval at which the sensor data 116 is communicated by the analyte monitoring device 104 may be different from the examples above without departing from the spirit or scope of the described techniques.

Additionally, the computing device 108 may maintain the sensor data 116 at least temporarily, e.g., in a storage device (not shown) of the computing device 108. The sensor data 116 may also be maintained in such storage of the computing device 108 or storage of a different device along with other associated data, such as with corresponding timestamps and/or identifiers of data packets in which communicated, to name just a few.

As depicted, the described systems may include the one or more computing devices 108 in accordance with the described techniques. In one or more scenarios, for instance, the described techniques may be implemented where the computing device 108 is a mobile device associated with the person 102, such as the person 102's mobile phone. Alternatively, the described techniques are implementable using multiple computing devices 108, which in at least one variation may include both a wearable device (e.g., a smart watch, mouthguard, contact lenses, smart glasses, chest strap, ear buds, or headphones, to name just a few) and a mobile phone. In such scenarios, both of these devices may be capable of performing at least some of the same operations, such as to receive the sensor data 116 from the analyte monitoring device 104 (and from other sources), generate sensor data 116 using onboard or associated sensors, communicate data via the network 114 to the glycemic control system 110 and/or the health monitoring platform 112, display information related to the data, display information related to recommendations produced by the glycemic control system 110 and/or the health monitoring platform 112, facilitate control of other devices (e.g., controlling the medicament delivery system 106), and so forth. Alternatively, or in addition, different devices may have different capabilities that other devices do not have or that are limited to specified devices through computing instructions.

The glycemic control system 110 causes therapy to be administered or recommended based on determinations made by processing the sensor data 116 and by providing instructions 118, such as by providing the instructions 118 to the medicament delivery system 106, the computing device 108, and/or to another device (e.g., an additional medicament delivery system). In accordance with the described techniques, the glycemic control system 110 makes those determinations and causes therapy to be administered or recommended, at least in part, by leveraging one or more of the computing devices 108, the analyte monitoring device 104, the medicament delivery system 106, and the glycemic control system 110.

In the illustrated example, the glycemic control system 110 is depicted including a medicament tracking engine 120 and a recommendation engine 122. It is to be appreciated that the glycemic control system 110 may include more, fewer, or different components without departing from the spirit or scope of the described techniques. Although depicted as being separate from the analyte monitoring device 104, the medicament delivery system 106, the computing devices 108, and the health monitoring platform 112, in one or more implementations, at least a portion of the glycemic control system 110 is implemented at one or more of those entities. Additionally, various portions of the glycemic control system 110 are implementable at or otherwise accessible using different combinations of the analyte monitoring device 104, the medicament delivery system 106, the computing devices 108, and the health monitoring platform 112 in accordance with the described techniques.

As discussed above and below, the glycemic control system 110 obtains the sensor data 116 from various sources. For example, the glycemic control system 110 obtains the sensor data 116 from one or more of the analyte monitoring device 104, the medicament delivery system 106, the computing devices 108, and the health monitoring platform 112. Examples of the sensor data 116 include, but are not limited to, global positioning system (GPS) data, Wi-Fi information (e.g., service set identifiers (SSIDs) of available networks, networks to which the computing device 108 is connected, and/or networks to which the computing device 108 previously connected), Bluetooth® Low Energy (BLE) information, data produced using a cellular antenna (e.g., long term evolution (LTE)), microphone data (e.g., sound data), accelerometer data, gyroscope data, magnetometer data, barometer data, ambient or internal temperature data (e.g., produced using temperature sensors), light data (e.g., captured using a device's camera), heart rate data (e.g., produced by a smartphone and/or a smart watch), proximity data (e.g., between devices), humidity data, analyte data, and medicament data, to name just a few. It is to be appreciated that this list is not exhaustive, and that the glycemic control system 110 may receive various other data (some of which is discussed above and below), without departing from the spirit or scope of the described techniques.

Based on the sensor data 116, the glycemic control system 110 determines the person 102's blood glucose level. In one or more implementations, the medicament tracking engine 120 logs or otherwise tracks medicament doses given by the medicament delivery system 106, including an amount and a time stamp of each dose (e.g., a date and time that the medicament was given). Additionally or alternatively, the medicament tracking engine 120 logs or otherwise tracks an amount of the medicament that is in the medicament delivery system 106 and available for delivery.

The recommendation engine 122 generates recommendations for one or more therapies to regulate blood glucose levels based on the sensor data 116. Examples of adverse effects include at least glucose excursions from a safe glucose range, such as hyperglycemia or hypoglycemia. In at least one implementation, the recommendation engine 122 suggests one or more doses of the medicament to deliver via the medicament delivery system 106. For example, in response to the measured glucose measurements measured by the analyte monitoring device 104 exceeding a pre-determined threshold, the recommendation engine 122 may calculate and suggest a bolus dose of the medicament. Additionally, or alternatively, the recommendation engine 122 may suggest multiple smaller doses of the medicament and a dosing interval for the multiple smaller doses.

In one or more implementations, the glycemic control system 110 provides the instructions 118 to notify the user about a recommended therapy. For instance, the glycemic control system 110 provides the instructions 118 to the computing device 108 to cause the computing device 108 to display or otherwise output the recommendation (or information indicative of the recommendation). Alternatively, or additionally, the glycemic control system 110 provides the instructions 118 to control a device (e.g., the medicament delivery system 106) to provide the recommended therapy. For instance, the glycemic control system 110 provides the instructions 118 to the medicament delivery system 106, and the instructions cause the medicament delivery system 106 to administer an amount of medicament to the person 102.

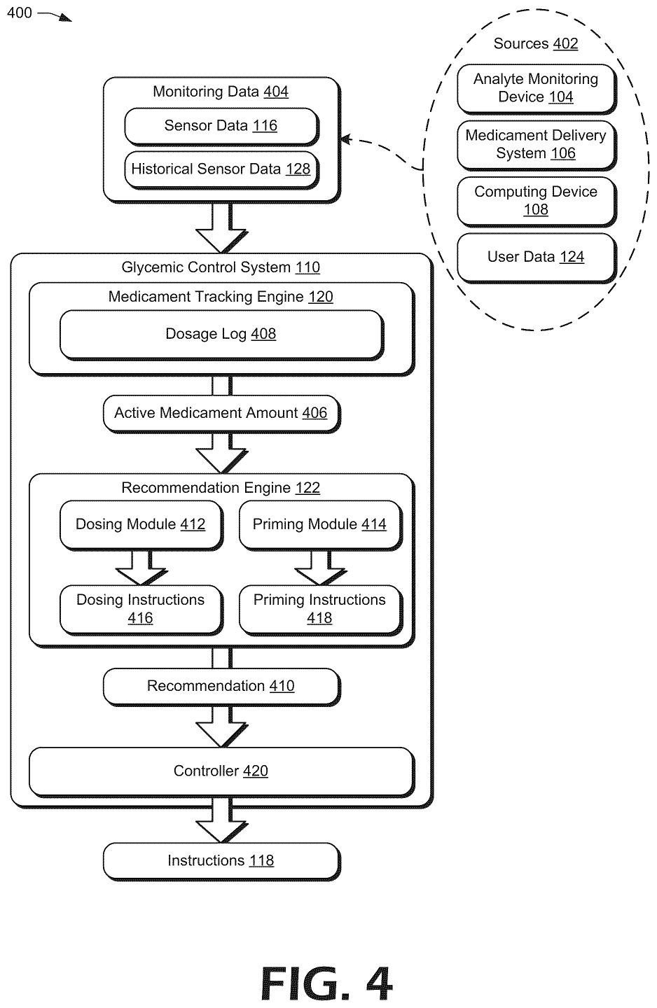

In one or more implementations, the instructions cause the medicament delivery system 106 to administer the medicament (e.g., a dose) to the person 102 automatically, without user input. In other implementations, the instructions instruct the medicament delivery system 106 to administer the medicament (e.g., a dose) to the person 102, but the system prevents the medicament from being delivered until a validation input is received from the user, such as an input indicating that the user allows the medicament to be delivered. Alternatively, or additionally, the instructions 118 instruct the medicament delivery system 106 to administer the dose of the medicament, and the medicament delivery system 106 utilizes user interaction to transfer the dose into the body of the person 102, such as when a user positions the medicament delivery system 106 against the person 102's body and actuates the system (e.g., presses a button) to administer the medicament. One example of the glycemic control system 110 is discussed in more detail below in relation to FIG. 4.

In one or more implementations, the glycemic control system 110 also leverages resources of the health monitoring platform 112 in connection with glycemic control. For instance, the health monitoring platform 112 may be configured to store data, such as the sensor data 116 (e.g., analyte measurements, data produced by other sensors, and data produced based on determinations made using the analyte measurements and/or data produced by other sensors), the instructions 118, user profile data associated with a user (e.g., the person 102), and/or user profile data associated with one or more other users of a user population (not shown). The environment 100 includes user data 124, which represents a variety of data obtained and stored by the health monitoring platform 112 about one or more of those users. Although stored about one or more users, the user data 124 may be obfuscated using one or more techniques, such that personally identifying information of the users associated with the data can be kept anonymous in various scenarios of using the data. In the illustrated environment 100, the user data 124 is shown stored in a storage device 126. The storage device 126 may represent one or more databases or other storage included as part of or otherwise accessible to the health monitoring platform 112. In accordance with the described techniques, the storage device 126 is capable of storing the user data 124 and various other data.

By way of example, the user data 124 may include any combination of the data discussed just above as well as other data discussed above and below. For instance, the user data 124 may include historical data associated with the one or more users, such as historical sensor data 128, determinations made based on the historical sensor data 128, user inputs received, and so on. In at least one implementation, such historical data may describe conditions of the users, therapies administered to the user, the responses of such therapies (e.g., changes to analyte measurements following administration of different doses), and so forth.

In one or more implementations, the health monitoring platform 112 includes a monitoring service 130. The monitoring service 130 may be used separately or in connection with the glycemic control system 110. By way of example, the monitoring service 130 may provide one or more web-based health-related services to users via the network 114 and their computing devices 108, e.g., mobile applications. The health monitoring platform 112 may include or have access to various computing resources, such as processing, storage, and virtual resources. Those resources may be useable, for example, to train, maintain, and/or deploy algorithms (e.g., machine learning algorithms), which may generate predictions associated with health monitoring by using the wealth of data collected about the person 102 and users of a user population. Thus, the monitoring service 130 may leverage those resources to execute algorithms and to implement other functionality in order to deliver web-based services to users via their devices. Notably, one or more such algorithms or functionalities may require an amount of computing resources that exceeds the resources of typical, personal computing devices, e.g., mobile phones, laptops, tablet devices, and wearables, to name just a few. Nonetheless, the health monitoring platform 112 may include or otherwise have access to at least a threshold amount of resources needed to operate such algorithms and provide such functionality, e.g., cloud storage, server devices, virtualized resources, and so forth. The health monitoring platform 112 may include a variety of resources that the computing devices 108 can leverage via the monitoring service 130 in order to provide user interfaces or generate data for location-aided glycemic control. In the context of measuring an analyte, e.g., glucose continuously, and obtaining analyte data describing such measurements, consider the following discussion of FIG. 2.

FIG. 2 depicts an example 200 of an implementation of the analyte monitoring device 104 of FIG. 1 in greater detail. In particular, the illustrated example 200 includes a top view and a corresponding side view of the analyte monitoring device 104. It is to be appreciated that the analyte monitoring device 104 may vary in implementation from the following discussion in various ways without departing from the spirit or scope of the described techniques.

In this example 200, the analyte monitoring device 104 is illustrated to include an analyte sensor 202 (e.g., a glucose sensor) and a sensor module 204. Here, the analyte sensor 202 is depicted in the side view having been inserted subcutaneously into skin 206, e.g., of the person 102. The sensor module 204 is approximated in the top view as a dashed rectangle. The analyte monitoring device 104 also includes a transmitter 208 in the illustrated example 200. Use of the dashed rectangle for the sensor module 204 indicates that it may be housed or otherwise implemented within a housing of the transmitter 208. Antennae and/or other hardware used to enable the transmitter 208 to produce signals for communicating data, e.g., over a wireless connection to the medicament delivery system 106 and/or the computing device 108, may also be housed or otherwise implemented within the housing of the transmitter 208. In this example 200, the analyte monitoring device 104 further includes adhesive pad 210.

In operation, the analyte sensor 202 and the adhesive pad 210 may be assembled to form an application assembly, where the application assembly is configured to be applied to the skin 206 so that the analyte sensor 202 is subcutaneously inserted as depicted. In such scenarios, the transmitter 208 may be attached to the assembly after application to the skin 206 via an attachment mechanism (not shown). Alternatively, the transmitter 208 may be incorporated as part of the application assembly, such that the analyte sensor 202, the adhesive pad 210, and the transmitter 208 (with the sensor module 204) can all be applied at once to the skin 206. In one or more implementations, this application assembly is applied to the skin 206 using a separate sensor applicator (not shown). Unlike the finger sticks required by conventional blood glucose meters, user-initiated application of the analyte monitoring device 104 with a sensor applicator is nearly painless and does not require the withdrawal of blood. Moreover, the automatic sensor applicator generally enables the person 102 to embed the analyte sensor 202 subcutaneously into the skin 206 without the assistance of a clinician or healthcare provider.

The analyte monitoring device 104 may also be removed by peeling the adhesive pad 210 from the skin 206. It is to be appreciated that the analyte monitoring device 104 and its various components as illustrated are simply one example form factor, and the analyte monitoring device 104 and its components may have different form factors without departing from the spirit or scope of the described techniques.

In operation, the analyte sensor 202 is communicably coupled to the sensor module 204 via at least one communication channel which can be a wireless connection or a wired connection. Communications from the analyte sensor 202 to the sensor module 204 or from the sensor module 204 to the analyte sensor 202 can be implemented actively or passively, and these communications can be continuous (e.g., analog) or discrete (e.g., digital).

The analyte sensor 202 may be a device, a molecule, and/or a chemical which changes or causes a change in response to an event which is at least partially independent of the analyte sensor 202. The sensor module 204 is implemented to receive indications of changes to the analyte sensor 202 or caused by the analyte sensor 202. For example, the analyte sensor 202 can include glucose oxidase, which reacts with glucose and oxygen to form hydrogen peroxide that is electrochemically detectable by the sensor module 204, which may include an electrode. In this example, the analyte sensor 202 may be configured as or include a glucose sensor configured to detect analytes in blood or interstitial fluid that are indicative of glucose level using one or more measurement techniques. In one or more implementations, the analyte sensor 202 may also be configured to detect analytes in the blood or the interstitial fluid that are indicative of other markers, such as lactate levels, ketones, or ionic potassium, which may increase an accuracy of identifying or predicting glucose-based events (e.g., hyperglycemia or hypoglycemia). Additionally or alternatively, the analyte monitoring device 104 may include additional sensors and/or architectures to the analyte sensor 202 to detect those analytes indicative of the other markers.

In another example, the analyte sensor 202 (or an additional sensor of the analyte monitoring device 104—not shown) can include a first and second electrical conductor, and the sensor module 204 can electrically detect changes in electric potential across the first and second electrical conductor of the analyte sensor 202. In this example, the sensor module 204 and the analyte sensor 202 are configured as a thermocouple such that the changes in electric potential correspond to temperature changes. In some examples, the sensor module 204 and the analyte sensor 202 are configured to detect a single analyte, e.g., glucose. In other examples, the sensor module 204 and the analyte sensor 202 are configured to use diverse sensing modes to detect multiple analytes, e.g., ionic sodium, ionic potassium, carbon dioxide, and glucose. Alternatively, or additionally, the analyte monitoring device 104 includes multiple sensors to detect not only one or more analytes (e.g., ionic sodium, ionic potassium, carbon dioxide, glucose, and insulin) but also one or more environmental conditions (e.g., temperature, moisture, movement). Thus, the sensor module 204 and the analyte sensor 202 (as well as any additional sensors) may detect the presence of one or more analytes, the absence of one or more analytes, and/or changes in one or more environmental conditions. As noted above, the analyte monitoring device 104 may be configured to produce data describing a single analyte (e.g., glucose) or multiple analytes.

In one or more implementations, the sensor module 204 may include a processor and memory (not shown). The sensor module 204, by leveraging the processor, may generate analyte measurements 212 based on the communications with the analyte sensor 202 that are indicative of the above-discussed changes. Based on the above-noted communications from the analyte sensor 202, the sensor module 204 is further configured to generate communicable packages of data that include at least one analyte measurement 212. In this example 200, the sensor data 116 represents these packages of data. Additionally, or alternatively, the sensor module 204 may configure the sensor data 116 to include additional data, including, by way of example, supplemental sensor information 214. The supplemental sensor information 214 may include a sensor identifier, a sensor status, temperatures that correspond to the analyte measurements 212, measurements of other analytes that correspond to the analyte measurements 212, and so forth. It is to be appreciated that supplemental sensor information 214 may include a variety of data that supplements at least one analyte measurement 212 without departing from the spirit or scope of the described techniques.

In implementations where the analyte monitoring device 104 is configured for wireless transmission, the transmitter 208 may transmit the sensor data 116 as a stream of data to a computing device (e.g., the computing device 108). Alternatively or additionally, the sensor module 204 may buffer the analyte measurements 212 and/or the supplemental sensor information 214 (e.g., in a memory of the sensor module 204 and/or other physical computer-readable storage media of the analyte monitoring device 104) and cause the transmitter 208 to transmit the buffered sensor data 116 later at various regular or irregular intervals, for example, time intervals (approximately every second, approximately every thirty seconds, approximately every minute, approximately every five minutes, approximately every hour, and so on), storage intervals (when the buffered analyte measurements 212 and/or supplemental sensor information 214 reach a threshold amount of data or a number of measurements), and so forth. It should be appreciated that in various implementations, the analyte monitoring device 104 can vary in numerous ways from the example described above without departing from the spirit or scope of the described techniques.

Having discussed one example of an analyte monitoring device, consider now the following discussion of one example medicament delivery system.

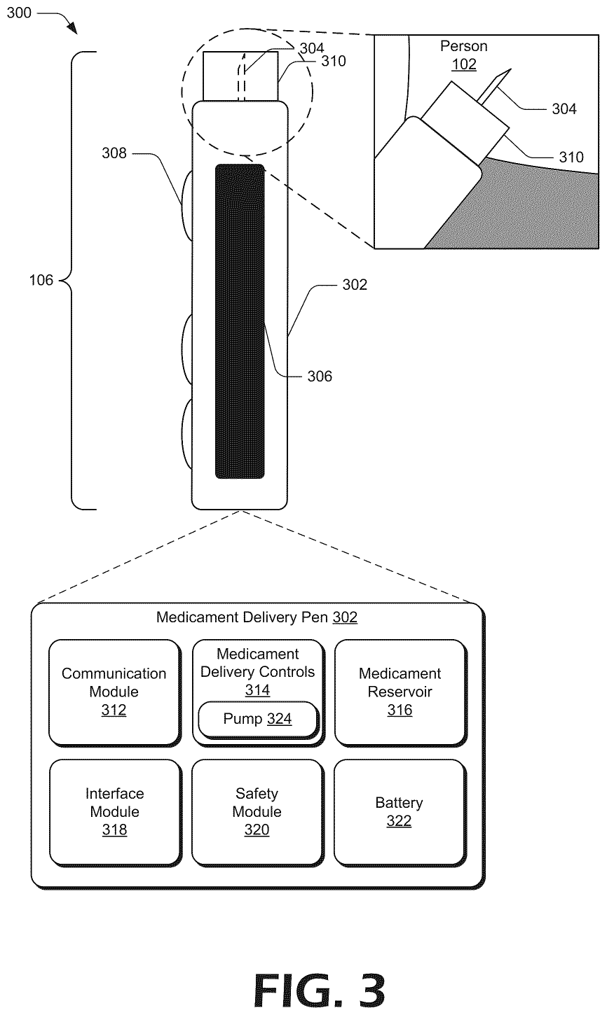

FIG. 3 depicts an example implementation 300 of the medicament delivery system 106 of FIG. 1 in greater detail. In the example implementation 300, the medicament delivery system 106 includes a medicament injection pen 302. Broadly speaking, the medicament injection pen 302 is a hand-held apparatus configured to subcutaneously deliver medicament into the person 102 for absorption by the person 102's bloodstream. In this way, the delivered medicament may be used by the person 102's body to maintain balanced analyte levels, e.g., within a target range of analyte measurements. In one or more implementations, the medicament injection pen 302 includes a needle 304 that is inserted subcutaneously into skin. Accordingly, the medicament injection pen 302 may administer doses of medicament through the skin of the person 102 when the needle 304 is inserted. As discussed herein, for instance, bolus doses of insulin may be administered through the skin of the person 102 via the needle 304. The medicament injection pen 302 may administer medicament to the person 102 based on user input that is received, for example, via a user interface 306 of the medicament injection pen 302, via at least one button 308 of the medicament injection pen 302, and/or via the computing device 108.

The medicament injection pen 302 involves user interaction (e.g., from the person 102 or a caretaker of the person 102) in order to administer medicament to the person 102. Such interaction may include, for example, interaction to position the medicament injection pen 302 where medicament stored in the pen can be administered to the person 102 and also interaction to initiate administration of the medicament. In one or more implementations, the medicament injection pen 302 includes the at least one button 308. The at least one button 308 may be programmed to receive one or more inputs. For example, a first of the at least one button 308 may be a power button configured to turn the medicament injection pen 302 “on” (e.g., in response to receiving input while the medicament injection pen 302 is powered off) or “off” (e.g., in response to receiving input while the medicament injection pen 302 is powered on). As another example, a second of the at least one button 308 may be a connection button configured to establish a wireless connection to one or more external devices, such as the analyte monitoring device 104 and/or the computing device 108. As yet another example, a third of the at least one button 308 may be a dosing button configured to administer the medicament to the person 102. In at least one implementation, the at least one button 308 is configured as an electronic button that sends an electrical signal to trigger an action, rather than a mechanical button that directly or indirectly performs the action via physical means.

It is to be appreciated that the at least one button 308 may be configured in a number of different other ways without departing from the spirit or scope of the described techniques. For example, in at least one implementation, a function of the at least one button 308 changes based on a type of input received (e.g., a short duration press, a long duration press, a sequence of presses). Additionally, or alternatively, the user interface 306 may indicate a current function of the at least one button 308. As another example, in some implementations, the medicament injection pen 302 does not include the at least one button 308, and user inputs are instead received via the user interface 306 and/or the computing device 108. The user interface 306 may include one or a combination of a display, a touchscreen (e.g., a touch-sensitive display), a vibrator for providing tactile (e.g., haptic) feedback, one or more buttons, and a speaker. In at least one implementation, the user interface 306 is configured to both output notifications (e.g., to the user) and receive input (e.g., from the user).

In at least one implementation, the needle 304 is retractable or otherwise housed internally within the medicament injection pen 302, such as within a cap 310, when the medicament injection pen 302 is not being used to administer medicament to the person 102. For example, the cap 310 may be removed (e.g., unfastened from a body of the medicament injection pen 302) to uncover the needle 304 and replaced (e.g., affixed to the body of the medicament injection pen 302) to cover the needle 304. As another example, the needle 304 and/or the cap 310 may include a retraction mechanism that is selectively actuated (e.g., via user input received via the user interface 306, the at least one button 308, the cap 310, and/or the computing device 108) to extend the needle 304 from the cap 310. For instance, the needle may be actuated to extend from the cap 310 in response to a first input and may be actuated to retract into the cap 310 in response to a second input. Furthermore, in at least one implementation, the needle 304 is replaceable.

In the example implementation 300, the medicament injection pen 302 includes a communication module 312, medicament delivery controls 314, a medicament reservoir 316, an interface module 318, a safety module 320, and a battery 322. In at least one implementation, the medicament injection pen 302 may be configured in various ways, such as with some of these components while others are housed or otherwise implemented in separate devices. Alternately or additionally, the medicament injection pen 302 may include additional or alternate components without departing from the spirit or scope of the techniques described herein.

The communication module 312 is configured to transmit data to and receive data from other devices, such as the computing device 108 and/or the analyte monitoring device 104. In one or more implementations, the communication module 312 establishes communicative couplings with such other devices to enable transmission and receipt of data. By way of example, the communication module 312 may establish, or otherwise facilitate establishing, links or channels of communication with those other devices. The links or channels may be configured in various ways, including but not limited to Bluetooth® (e.g., BLE links), NFC, 5G or other cellular, and Wi-Fi, to name just a few. Such communicative couplings enable the medicament injection pen 302 to communicate over different networks, such as the network 114 and/or securely within a system including, for example, the analyte monitoring device 104 and at least one computing device 108.

Once a communicative coupling is established, the communication module 312 can cause data to be transmitted over the established coupling and/or can receive data from other devices over the established coupling. Additionally, or alternatively, the communication module 312 may be configured to establish connections over wired communication channels-such as via a cord (e.g., a USB cord) connected to the medicament injection pen 302 and another device—and also configured to transmit and/or receive data over such a wired coupling. The communication module 312 may be configured in various ways to enable the medicament injection pen 302 to communicate with other devices.

As one example, the communication module 312 enables the medicament injection pen 302 to receive the instructions 118 and/or other instructions from the computing device 108 and/or the glycemic control system 110 for controlling delivery of medicament to the person 102. For instance, the communication module 312 enables the medicament injection pen 302 to receive instructions that instruct the medicament injection pen 302 regarding delivery of a bolus dose of insulin to the person 102 (e.g., an amount to bolus within a limited time), multiple doses of insulin to provide to the person 102 at pre-determined intervals, and so forth. The computing device 108 may send a variety of communications to the medicament delivery system 106 for controlling medicament delivery without departing from the spirit or scope of the techniques described herein. Alternatively, the medicament injection pen 302 determines the medicament delivery instructions without additional instructions from the computing device 108, such as when the glycemic control system 110 is included in the medicament injection pen 302.

The medicament delivery controls 314 represent any hardware, software, and/or mechanical components of the medicament injection pen 302 that cause medicament to be pumped (or otherwise extracted) from the medicament reservoir 316 so that it flows through the needle 304 and into the person 102. Moreover, the medicament delivery controls 314 are further configured to cause the medicament to be pumped or otherwise extracted from the medicament reservoir 316 in accordance with medicament dose instructions, e.g., the instructions 118 from the glycemic control system 110 which specify one or more deliveries of the medicament.

In the illustrated implementation 300, the medicament delivery controls 314 include a pump 324. In at least one implementation, the pump 324 is a motorized pump. For example, the pump 324 may include a stepper motor, wherein each rotation of the stepper motor is divided into a pre-defined number of steps. In such examples, each step extracts a known quantity of the medicament from the medicament reservoir 316 for delivery via the needle 304, and the medicament delivery controls 314 may calculate the number of steps to rotate the stepper motor in order to extract a desired amount of the medicament.

For example, in at least one implementation, the medicament delivery controls 314 determine pump control instructions based on the instructions 118. The pump control instructions may define operation parameters that will cause the pump 324 to provide the desired amount of the medicament. The operation parameters may include, for example, an amount or pulse-width of voltage to supply to the pump 324 (e.g., from the battery 322). As a non-limiting example, the medicament delivery controls 314 may transmit a control signal to power the pump 324 for a duration that is in proportion to the desired amount of the medicament.

The medicament reservoir 316 is configured to house an amount of the medicament, which may be subcutaneously delivered via the needle 304 by leveraging the functionality of the medicament injection pen 302. The medicament reservoir 316 may be replaceable or otherwise configured so that the amount of medicament can be replenished in the medicament reservoir 316. As an example, the medicament reservoir 316 may be a pre-filled cartridge that is inserted into medicament injection pen 302 when in a “full” state (e.g., when the amount of the medicament held therein is greater than a pre-determined upper threshold) and removed from the medicament injection pen 302 when in an “empty” state (e.g., when the amount of the medicament held therein is less than a pre-determined lower threshold). At least during injection, the medicament reservoir 316 may be fluidically coupled to the pump 324 and the needle 304 so that operation of the pump 324 causes the medicament to flow from the medicament reservoir 316 through the needle 304. The medicament reservoir 316 may be configured in various ways (e.g., different shapes, different materials, differently removable, and so on) without departing from the spirit or scope of the described techniques.

In some implementations, the medicament injection pen 302 may be configured to identify a type of the medicament in the medicament reservoir 316 based on, for example, markings or other indicators on the medicament reservoir 316. By way of example, the medicament injection pen 302 may include a surface mount technology (SMT) resistor that detects a resistor value of a pre-filled cartridge when the pre-filled cartridge is inserted. In such examples, the resistor value may be different for different types of the medicament, and the medicament delivery controls 314 may be programmed to associate the different resistor values with the corresponding type of the medicament. Different types of the medicament may have different durations of action (e.g., long-acting insulin versus rapid-acting insulin), concentrations, or active components, for example, which may affect the dosing. Accordingly, in one or more implementations, the medicament injection pen 302 detects the type of medicament in the medicament reservoir 316 and communicates the type of medicament to the glycemic control system 110 (e.g., via the communication module 312).

The interface module 318 is configured to cause display of information via the user interface 306 of the medicament injection pen 302. The interface module 318 may generate one or more user interfaces for display via the user interface 306. By way of example, the interface module 318 may cause display of a user interface for setting up a wireless connection with the computing device 108 via the user interface 306. Additionally or alternatively, the interface module 318 may cause the user interface 306 to display analyte measurements (e.g., in a similar manner as they are displayed via a health monitoring application of the computing device 108), trend arrows (e.g., regarding an identified trend in the analyte measurements), alerts (e.g., about the analyte measurements, other physiological conditions, operability of the medicament delivery system 106, operability of components of the glycemic control system 110, status of a wireless connection with the computing device 108, and so on), set up interfaces, indications of being out of communication range from different devices (e.g., the computing device 108 or the analyte monitoring device 104), and so forth. By way of example, the user interface 306 may display or otherwise communicate (e.g., via audible and/or tactile alerts) a series of notifications indicating a current status of the medicament injection pen 302, such as a suggested dose of the medicament to be administered, an indication that dosing is in progress, and an indication that dosing is complete. The interface module 318 may thus cause a variety of information to be communicated via the user interface 306 of the medicament injection pen 302.

The interface module 318 may also be configured to receive information via the user interface 306 and/or the at least one button 308. By way of example, the interface module 318 may receive user input regarding setting up the wireless connection with the computing device 108, receive commands to operate the medicament injection pen 302, receive user preferences, and so forth. The interface module 318 may thus receive a variety of information via the user interface 306 and/or the at least one button 308.

Safety module 320 is configured to provide one or more safeguards to control delivery of medicament so that the delivery is not harmful (e.g., so that the medicament is delivered safely). By way of example, the safety module 320 may contain or otherwise enforce delivery limits, such as maximum and minimum doses over different periods of time. These limits are effective to prevent erroneous delivery instructions from the glycemic control system 110 from affecting the person 102 in an adverse way, such as when an error transmitting the instructions affects their contents or when an error in a prediction made by one or more machine learning models affects those instructions. For instance, the safety module 320 may limit an amount or rate of medicament that the medicament injection pen 302 delivers to a threshold amount or threshold rate, even if instructions received from the glycemic control system 110 instruct the medicament injection pen 302 to deliver more than the threshold amount. Similarly, the safety module 320 may also prevent the medicament injection pen 302 from delivering less than a threshold amount of medicament or delivering medicament at less than a threshold rate, even if instructions received from the glycemic control system 110 instruct the medicament injection pen 302 to deliver less than the threshold amount.

In addition, the safety module 320 is configured to continue operating the medicament injection pen 302 in the absence of instructions from the glycemic control system 110 or the computing device 108, e.g., in the absence of instructions describing how much medicament to deliver and when. The safety module 320 may be configured to continue operating the medicament injection pen 302 to deliver medicament to the person 102 when the glycemic control system 110 is out of communication range, for example. The safety module 320 may have access to logic, default settings, or manual settings entered by the user, to name just a few, that control how much medicament the medicament injection pen 302 is to deliver when instructions are not being received from the glycemic control system 110. The safety module 320 may perform various additional or different safeguards to ensure that an amount of medicament delivered is not harmful to the person 102.

The battery 322 is configured to provide power for operating the medicament injection pen 302, such as to power the communication module 312 to send and receive data, to power the medicament delivery controls 314 to cause delivery of medicament from the medicament reservoir 316 to the person 102 via the needle 304, to power the interface module 318 to display information via the user interface 306, and so forth. The battery 322 may be rechargeable (e.g., via a charging port or wirelessly) or replaceable. It is to be appreciated that the battery 322 may be configured in various ways.

Although not depicted, the medicament delivery system 106 or another device (e.g., the analyte monitoring device 104) may be configured with a medicament sensor (e.g., an insulin sensor). Such a medicament sensor may be applied to skin or inserted subcutaneously to measure a systemic level of the medicament, e.g., in the person 102. Accordingly, the medicament sensor may be included as part of the needle 304, the analyte monitoring device 104, or may be separately applied. In any case, such a sensor may be used in connection with the safety module 320 and/or with dose prediction functionality of the glycemic control system 110. In this way, medicament measurements produced using the medicament sensor can be used to prevent or detect an overdose of the medicament, such as to prevent a person who is receiving insulin from experiencing an episode of hypoglycemia.

By way of example, the safety module 320 may shut off medicament delivery by the medicament injection pen 302 based on the medicament measurements. For example, if the safety module 320 detects that the level of medicament surpasses a predetermined threshold, the safety module 320 may prevent the medicament delivery controls 314 from delivering additional medicament. Based on the medicament measurements, the glycemic control system 110 may additionally or alternatively send instructions to the medicament delivery system 106 instructing it to cease medicament delivery. In one or more implementations, the safety module 320 and/or the computing device 108 may also trigger alerts if the level of medicament surpasses the predetermined threshold. Physiologically, different thresholds may be determined for different persons based on their medicament sensitivities (e.g., insulin sensitivities), such that the above-discussed shut down, alerts, or ceases to medicament delivery may be triggered at different medicament levels for the different persons.

Additionally, or alternatively, the medicament delivery controls 314 and/or the safety module 320 may track an amount of medicament that has been delivered to the person 102 during a specific timeframe in which the medicament can provide glycemic control. By way of example, the tracked medicament may be “active” medicament that is expected to be working in the person 102's body (e.g., a quantity termed “insulin on board”). For example, the medicament delivery controls 314 and/or the safety module 320 may log all dosing provided by the medicament injection pen 302, which may be communicated to the glycemic control system 110 (e.g., to the medicament tracking engine 120). The glycemic control system 110 may use such information in determining subsequent doses, such as when at least a portion of a previously provided dose is predicted to still be active (e.g., based on an amount of time that has passed since the previously provided dose was administered).

In this way, the amount of medicament delivered by the pump 324 is controlled (e.g., by the medicament delivery controls 314) based on the instructions 118 communicated by the glycemic control system 110 in combination with the safety module 320 and without relying on user input. As a result, administered medicament amounts may be more easily and accurately tracked (e.g., by the medicament tracking engine 120), which may result in more accurate calculations for subsequent doses by the glycemic control system 110. Furthermore, by eliminating the need for user input into the medicament injection pen 302 regarding the medicament dose to deliver, a speed at which the medicament injection pen 302 can be operated to provide the calculated medicament dose is increased.

Having considered an example of an environment and example devices, consider now a discussion of some examples of details of the techniques for automatic glycemic control in accordance with one or more implementations.

### Automatic Medicament Dosing

FIG. 4 depicts an example of a system 400 that receives and processes sensor data from various sources to generate recommendations for medicament doses to deliver (e.g., via the medicament delivery system 106) for glycemic control. The illustrated system 400 includes, from FIG. 1, the glycemic control system 110 having the medicament tracking engine 120 and the recommendation engine 122.

In this example, the glycemic control system 110 is depicted obtaining monitoring data 404, including the sensor data 116 and the historical sensor data 128. In accordance with the described techniques, the glycemic control system 110 receives the monitoring data 404 from various sources 402. The analyte monitoring device 104, the medicament delivery system 106, and the computing device 108 may be the sources 402 of such data. Alternatively, or in addition, the glycemic control system 110 receives the monitoring data 404 from other sources 402, which may include or be associated with one or more various types of sensors. Furthermore, the glycemic control system 110 may receive other types of data from the various sources 402 in addition to the sensor data 116 and the historical sensor data 128, such as user preference data specifying, for example, medicament dosing preferences (e.g., a single larger bolus, multiple smaller boluses given at intervals, etc.).

In at least one implementation, the medicament tracking engine 120 predicts an active medicament amount 406 currently in a user (e.g., the person 102) based on a dosage log 408. For example, the dosage log 408 stores information regarding a date, time, and amount of each dose provided by the medicament delivery system 106, and the medicament tracking engine 120 may utilize this information in combination with information regarding a duration of action by the medicament in the user's body (e.g., a duration of insulin action). For example, different individuals may have different durations of action (e.g., how quickly or slowly each person's body uses up the delivered medicament). The duration of action may further vary based on a type of medicament given. For example, long-acting insulin and rapid-acting insulin have different activity profiles regarding how fast and how long they act to help regulate blood glucose. Furthermore, in at least one implementation, the medicament tracking engine 120 utilizes the sensor data 116 and/or the historical sensor data 128 to estimate the user's response to previously provided doses of the medicament. Additionally, or alternatively, the sensor data 116 may include a measurement of the active medicament amount 406. It is to be appreciated that the medicament tracking engine 120 may include or otherwise have access to various types of logic to predict the active medicament amount 406 currently in the user (e.g., the person 102) based on the sensor data 116, the historical sensor data 128, and/or the dosage log 408 in accordance with the described techniques. In this example, the medicament tracking engine 120 outputs the active medicament amount 406.

The system 400 determines a recommendation 410 via the recommendation engine 122. Here, the recommendation engine 122 is depicted receiving the medicament amount 406. It may be understood that is various scenarios, the active medicament amount 406 is a non-zero value (e.g., a number of insulin units), while in other scenarios, the active medicament amount 406 is zero (e.g., there is no active insulin from a previous administration currently in effect in the person 102). In the illustrated example, the recommendation engine 122 includes a dosing module 412 and a priming module 414. It is to be appreciated that the recommendation engine 122 may include more, different, or fewer modules without departing from the spirit or scope of the described techniques.

The dosing module 412 may include or otherwise have access to various types of logic (e.g., algorithms) to calculate one or more doses of the medicament to provide to the user, which may be output as dosing instructions 416. The dosing module 412 may calculate the one or more doses based on, for example, the active medicament amount 406, a current blood glucose level of the user (e.g., as determined based on the sensor data 116), and/or a rate of change in the blood glucose level. The dosing module 412 may further calculate the one or more doses based on the type of medicament to be injected (e.g., the type of medicament in the medicament reservoir 316). The type of medicament may include information regarding a manufacturer, concentration, duration of action, and active components of the medicament to be injected. By way of example, the dose may be calculated differently for long-acting insulin compared with rapid-acting insulin.

In accordance with the described techniques, the recommendation 410 output by the recommendation engine 122 may include the dosing instructions 416 that are determined by the dosing module 412. The dosing instructions 416 may include a number of doses, a frequency of the doses, and an amount of medicament in each dose. In one or more variations, the number of doses varies based on user preferences stored in the user data 124. As mentioned above, some users may prefer fewer needle sticks and may thus prefer receiving a single bolus dose of the medicament at a given time. In other examples, the user may prefer multiple smaller injections of the medicament over a period of time in order to avoid large changes in blood glucose levels upon each injection. Due to the different dosing schedule of the multiple smaller injections, the multiple smaller injections may result in more or less of the medicament being delivered compared with providing a single larger dose, at least in some examples. Additionally, or alternatively, the dosing module 412 may determine whether to recommend a single larger bolus injection or multiple smaller bolus injections based on the monitoring data 404 in order to provide effective glycemic control using one or more algorithms (e.g., machine learning algorithms) stored therein.

The dosing module 412 may further take into account a medicament sensitivity (e.g., an insulin sensitivity) of the user in determining the dosing instructions 416, as users who are less sensitive to the medicament may need higher doses of the medicament to regulate their blood glucose levels compared with users who are more sensitive to the medicament. In at least one implementation, the dosing module 412 estimates the medicament sensitivity of the user based on the historical sensor data 128. For example, the dosing module 412 may estimate the medicament sensitivity based on how much the user's blood glucose level decreased per unit of medicament given according to the historical sensor data 128 and the dosage log 408. Additionally, or alternatively, the medicament sensitivity may be calculated separately (e.g., by the user or a healthcare professional associated with the user) and input as part of the monitoring data 404.

The priming module 414 may include or otherwise have access to various types of logic to determine whether or not priming is indicated, and if indicated, an amount of the medicament to administer to prime the medicament delivery system 106. When priming is indicated, the amount of medicament to administer to prime the medicament delivery system 106 is output as priming instructions 418. As used herein with respect to the medicament delivery system 106, the term “prime” denotes removing air bubbles from the medicament delivery system 106 (e.g., from the needle 304) and ensures that the medicament delivery system 106 is open and working for medicament administration. In at least one implementation, priming is indicated when it has been at least a threshold duration (e.g., a number of minutes or hours) since a preceding (e.g., most recent prior) administration of the medicament. For example, it may be expected that the needle remains filled with the medicament when less than the threshold duration has elapsed since the preceding administration.

Additionally, or alternatively, the amount of medicament indicated in the priming instructions 418 may vary based on an elapsed duration since the preceding administration. For example, the amount indicated may vary between zero (e.g., when priming is not indicated) and a maximum priming value (e.g., when no medicament is expected to be in the needle 304). As another example, the priming instructions 418 may include the maximum priming value when a new (e.g., unused) needle 304 is attached to the medicament injection pen 302, which may be input to the glycemic control system 110 as part of the monitoring data 404. It is to be appreciated that the priming module 414 may determine the priming instructions 418 in a variety of other ways without departing from the spirit or scope of the described techniques.

The recommendation engine 122 generates the recommendation 410 based on the dosing instructions 416 generated by the dosing module 412 and the priming instructions 418 generated by the priming module 414. In one or more implementations, the recommendation 410 is generated in response to the user's blood glucose level increasing above a pre-determined threshold, as determined based on the sensor data 116. As an example, the pre-determined threshold may correspond to an elevated blood glucose level (e.g., hyperglycemia) at or above which the user may experience adverse effects. The recommendation engine 122 then generates the recommendation 410 to provide a mitigating therapy and/or notification of the mitigating therapy to the user (e.g., the person 102). The recommendation 410 may include one or more of a variety of therapies to control the user's blood glucose level. By way of example, and not limitation, the recommendation 410 may include the dose and/or timing of one or more medicaments to deliver via the medicament delivery system 106, activities to perform (e.g., exercise, contacting a health care professional, discontinuing an activity, resting) and/or timing of those activities, amount and/or timing of one or more foods to consume (e.g., drink a number of ounces of juice or eat a tablet), and so on. The recommendation 410 may recommend a variety of therapies without departing from the spirit or scope of the described techniques.

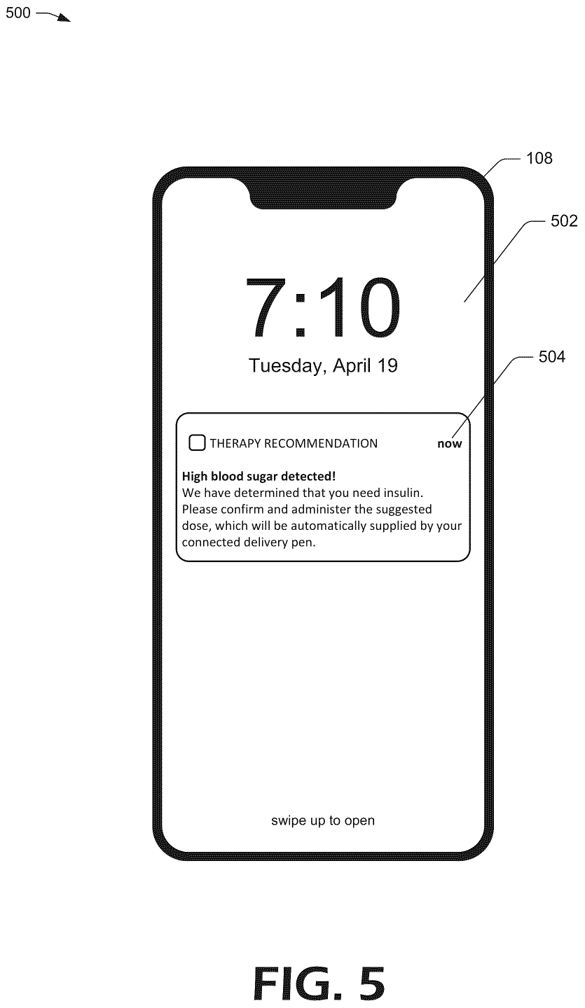

Based on the recommendation 410, a controller 420 outputs the instructions 118. For example, the controller 420 provides the instructions 118 to the medicament delivery system 106, the computing device 108, and/or to another device (e.g., an additional medicament delivery system). In one or more implementations, the controller 420 provides the instructions 118 to notify the user about the recommendation 410. For instance, the controller 420 provides the instructions 118 to the computing device 108 to cause the computing device 108 to display or otherwise output the recommendation 410 (or information indicative of the recommendation 410). Example notifications that can be output by the computing device 108 and/or the medicament delivery system 106 are discussed in more detail in relation to FIGS. 5-7.

Alternatively, or additionally, the controller 420 provides the instructions 118 to control a device (e.g., the medicament delivery system 106) to provide the recommended mitigating therapy. For instance, the controller 420 provides the instructions 118 to the medicament delivery system 106, and the instructions cause the medicament delivery system 106 to administer an amount of the medicament to the person 102 according to the dosing instructions 416. The instructions 118 may further include the priming instructions 418 so that the medicament delivery system 106 is prepared for providing the indicated amount of the medicament.

In one or more implementations, the instructions 118 cause the medicament delivery system 106 to administer the medicament (e.g., a dose of the medicament) to the person 102 with reduced user input. For example, the instructions 118 instruct the medicament delivery system 106 to administer the medicament (e.g., the dose) to the person 102, but the system prevents the medicament from being delivered until a validation input is received from the user. The validation input may be an input indicating that the user allows the medicament to be delivered. Alternatively, or additionally, the instructions 118 instruct the medicament delivery system 106 to administer the dose of the medicament, and the medicament delivery system 106 relies on user interaction to transfer the dose into the body of the person 102. For example, the user is instructed (e.g., via the instructions 118) to position the medicament delivery system 106 against the person 102's body and actuate the system (e.g., depress the at least one button 308) to administer the medicament.

FIG. 5 depicts an example of a user interface displaying an intervention notification. The illustrated example 500 includes, from FIG. 1, an example of the computing device 108 displaying an example user interface 502 via a display device, e.g., a touchscreen. Here, the user interface 502 includes an intervention notification 504, which indicates that a therapy is recommended for controlling a user's high blood sugar (e.g., hypoglycemia).

In this example, the high blood sugar is detected based on the sensor data 116 indicating that the blood glucose level of the user (e.g., the person 102) is greater than a pre-determined hyperglycemia threshold. The intervention notification 504 also the instructions 118, which in this example, instruct the user to confirm and administer a suggested dose(s) of medicament (e.g., insulin) via the user's connected delivery pen (e.g., the medicament injection pen 302). For example, insulin lowers the user's glucose levels, and thus, the suggested dose is predicted to bring the user's blood glucose levels below the hyperglycemia threshold without decreasing them below a pre-determined hypoglycemia threshold. In accordance with the techniques described herein, one or more suggested doses are determined via the recommendation engine 122 and automatically supplied by the medicament injection pen 302 in response to the user confirming and administering each dose, without the user manually setting each dose at the medicament injection pen 302.

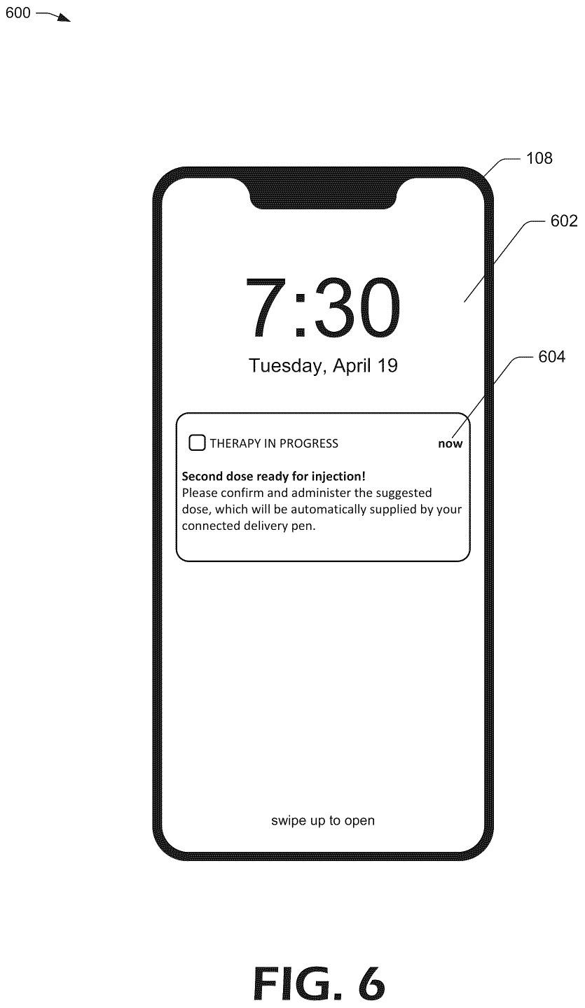

FIG. 6 depicts an additional example of a user interface displaying an intervention notification. Like the illustrated example 500, the illustrated example 600 includes, from FIG. 1, an example of the computing device 108 displaying an example user interface 602 via a display device, e.g., a touchscreen. Here, the user interface 602 includes an intervention notification 604 that indicates that a second dose is ready for injection. Intervention notification 604 corresponds to a second notification displayed subsequent to the intervention notification 504 of FIG. 5, which again instructs the user to confirm and administer a suggested dose of medicament (e.g., insulin) via the user's connected delivery pen. For example, the intervention notification 604 may be output in response to the user receiving a sequence of smaller medicament doses. Accordingly, the intervention notification 604 is output after the user administers a first dose of the medicament via the medicament injection pen 302 and when it is time to administer the second dose. It is to be appreciated that additional intervention notifications may be output via the computing device 108 to instruct the user to administer one or more additional doses according to the instructions 118 output by the glycemic control system 110. Furthermore, the intervention notification 504 and/or the intervention notification 604 may include audible and/or tactile components in addition to or as an alternative to text-based messages.

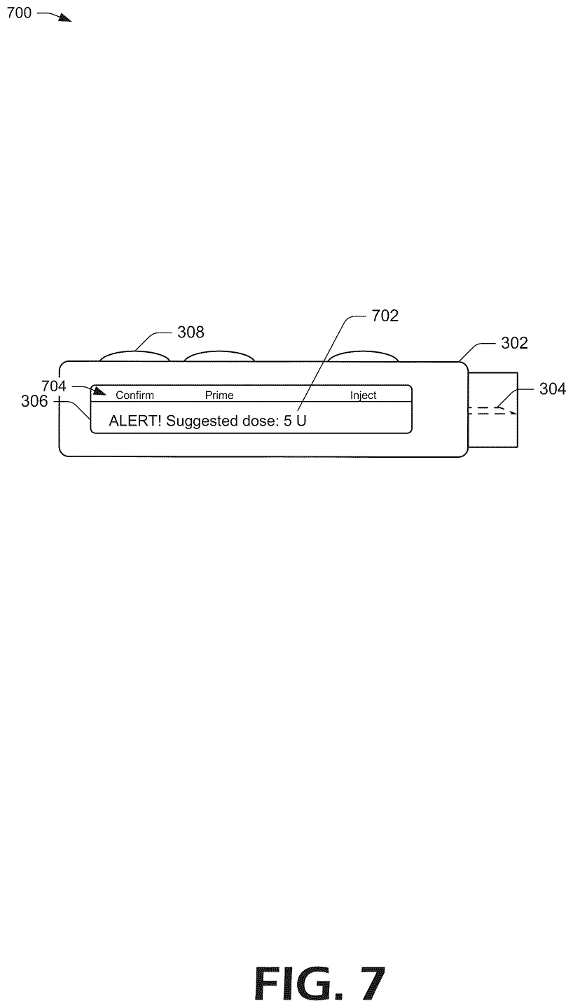

FIG. 7 depicts an additional example of a user interface displaying an intervention notification. The illustrated example 700 includes an example of the medicament injection pen 302 of FIG. 3 displaying an intervention notification 702 on the user interface 306. The intervention notification 702 indicates that a medicament dose is suggested for injection. The user interface 306 further displays button control indicators 704 that show an assigned function of the at least one button 308, such as when the function of the at least one button 308 can vary. In the depicted example, the button control indicators 704 indicate that a first button is programmed to receive user input confirming the suggested dose, a second button is programmed to receive user input to prime the needle 304, and a third button is programmed to receive user input to administer the injection of the medicament (e.g., the suggested dose of the medicament). Similar to the illustrated examples 500 and 600, the intervention notification 702 may include audible and/or tactile components in addition to or as an alternative to the text-based message.

FIG. 8 depicts an example 800 of a first combination of devices for implementing automatic medicament dosing. The illustrated example 800 includes the analyte monitoring device 104, the medicament delivery system 106, and the computing device 108 having the glycemic control system 110. In the illustrated example 800, the medicament delivery system 106 includes two delivery devices, including a medicament pump 802 as a first delivery device and the medicament injection pen 302 as a second delivery device. The medicament pump 802 is a wearable pump (e.g., an insulin pump) having an infusion set 804 that is applied to the person 102 (not shown in FIG. 8) to deliver medicament via the infusion set 804 at an insertion site. In one or more implementations, the infusion set 804 includes a cannula that is inserted subcutaneously into skin to deliver the medicament. Although the infusion set 804 is depicted with tubing 806 connected to the medicament pump 802, in one or more implementations, the infusion set 804 is tubeless.

In accordance with the described techniques, the analyte monitoring device 104, the medicament delivery system 106, and the computing device 108 may be communicably coupled, e.g., over the network 114 or via some other wireless connection (e.g., BLE), to implement any of the glycemic control techniques described above and below. In at least one implementation, only one of the medicament pump 802 and the medicament injection pen 302 is communicably connected to the computing device 108 and/or the analyte monitoring device 104 at a given time. For example, the medicament injection pen 302 may be utilized as the medicament delivery system 106 when the person 102 is not wearing the medicament pump 802, when the medicament pump 802 is powered down, and/or when the medicament pump 802 is not in communication with the computing device 108 and/or the analyte monitoring device 104. By way of example, the person 102 may discontinue wearing the medicament pump 802 for a period of time due to, for example, tiring of wearing the medicament pump 802 or expenses involved with the medicament pump 802. During that period of time, the person 102 may instead utilize the medicament injection pen 302 to administer the medicament.

In at least one variation, both of the medicament pump 802 and the medicament injection pen 302 are communicably connected to the computing device 108 and the analyte monitoring device 104 and both utilized to provide medicament delivery. By way of example, the medicament pump 802 may provide basal doses of the medicament, and the medicament injection pen 302 may provide bolus doses of the medicament (e.g., before meals). Thus, the combination of devices in the example 800 may be utilized in a variety of ways.

In one or more implementations, the analyte monitoring device 104, the medicament delivery system 106, and the computing device 108 are operably connected to implement glycemic control as a closed-loop system, an open-loop system, or a partially open-loop system. As a closed-loop system, the analyte monitoring device 104, the medicament delivery system 106, and the computing device 108 are configured to monitor an analyte of the person 102 wearing the analyte monitoring device 104, recommend therapies based on an activity that the person 102 is predicted to participate in at a predicted location, and administer the recommended therapy (e.g., via the medicament delivery system 106) without user interaction. By way of example, the medicament pump 802 may be worn by the person 102 to implement the medicament delivery system 106 as the closed-loop system.

By way of contrast, in a partially open-loop system, a user may be required to validate a therapy before it is administered by the system. For example, the user may be required to provide approval of a recommended medicament dose (e.g., recommended by the glycemic control system 110) via a display of the medicament delivery system 106 or the computing device 108 before the medicament delivery system 106 automatically prepares the recommended medicament dose for administration. Once validated, the medicament delivery system 106 may administer the recommended dose, either with or without additional user interaction. By way of example, when the medicament pump 802 is used to provide the recommended medicament dose, the infusion set 804 may already be subcutaneously inserted at the insertion site. In such an example, the medicament pump 802 may provide the recommended medicament dose in response to receiving approval from the user. In contrast, when the medicament injection pen 302 is used to provide the recommended medicament dose, the user may confirm that the needle 304 has been inserted, such as by depressing the at least one button 308 to initiate administering the recommended medicament dose.

In an open-loop system, the glycemic control system 110 may simply cause a recommended therapy to be output (e.g., displayed) via the computing device 108 (e.g., a display of the computing device 108). In order to administer a recommended dose of the medicament in an open system, the user may provide input to the medicament delivery system 106 specifying the recommended medicament dose and select a control (e.g., a displayed control) to cause the medicament delivery system 106 to administer the dose. Thus, the user manually sets the dosage delivered by the medicament delivery system 106 in the open-loop system, which is in contrast to the automatic dosing of the partially open-loop system described above. By way of example, a non-automatic injection pen may be limited to operating in the open-loop configuration. The medicament pump 802 and the medicament injection pen 302 may also be operated in the open-loop configuration, if desired. However, the partially open-loop system reduces user interaction and reduces potential sources of user error compared with the open-loop system. As such, the automatic medicament delivery enabled by the first combination of devices advantageously decreases adverse health effects associated with hyperglycemia and hypoglycemia by providing highly accurate medicament dosing and also reduces a burden on the user to determine and administer a correct dose.

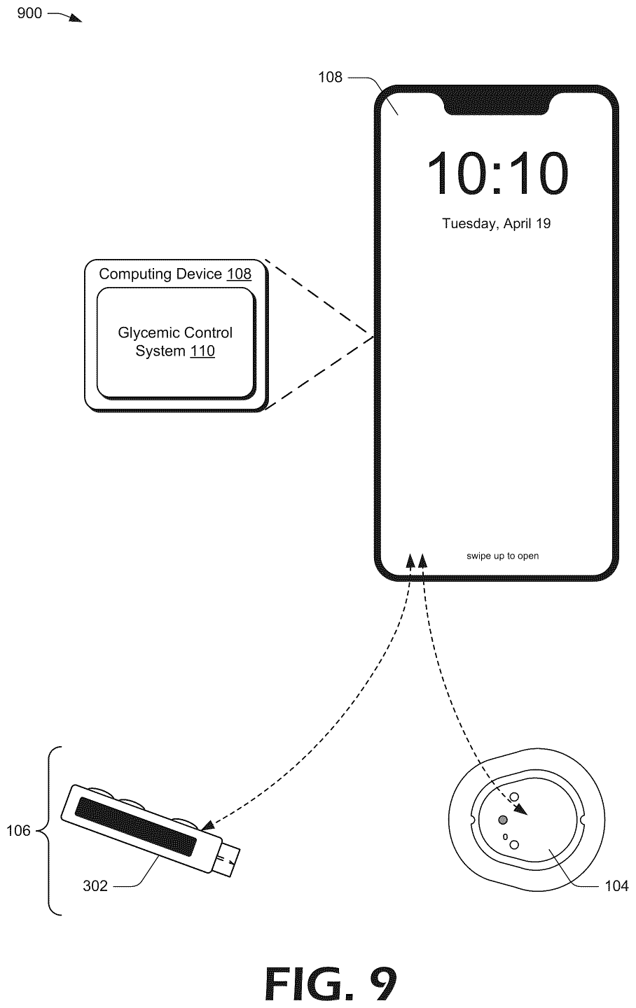

FIG. 9 depicts an example 900 for implementing automatic medicament dosing. The illustrated example 900 includes the analyte monitoring device 104, the medicament delivery system 106, and the computing device 108 having the glycemic control system 110. In contrast to the example 800, though, in the illustrated example 900, the medicament delivery system 106 includes only the medicament injection pen 302 rather than both the medicament injection pen 302 and the medicament pump 802.

Since the medicament delivery system 106 includes the medicament injection pen 302 (and not the medicament pump 802), the system in the illustrated example 900 necessarily involves user interaction. Such interaction may include, for example, interaction to position the medicament injection pen 302 to administer medicament stored in the medicament injection pen 302 to the person 102 (not shown in FIG. 9) and also interaction to initiate administration of the medicament (e.g., by depressing the at least one button 308). Thus, when the analyte monitoring device 104, the medicament delivery system 106, and the computing device 108 are configured as in the example 900, those devices can be operably connected to implement glycemic control as an open-loop system or a partially open-loop system.

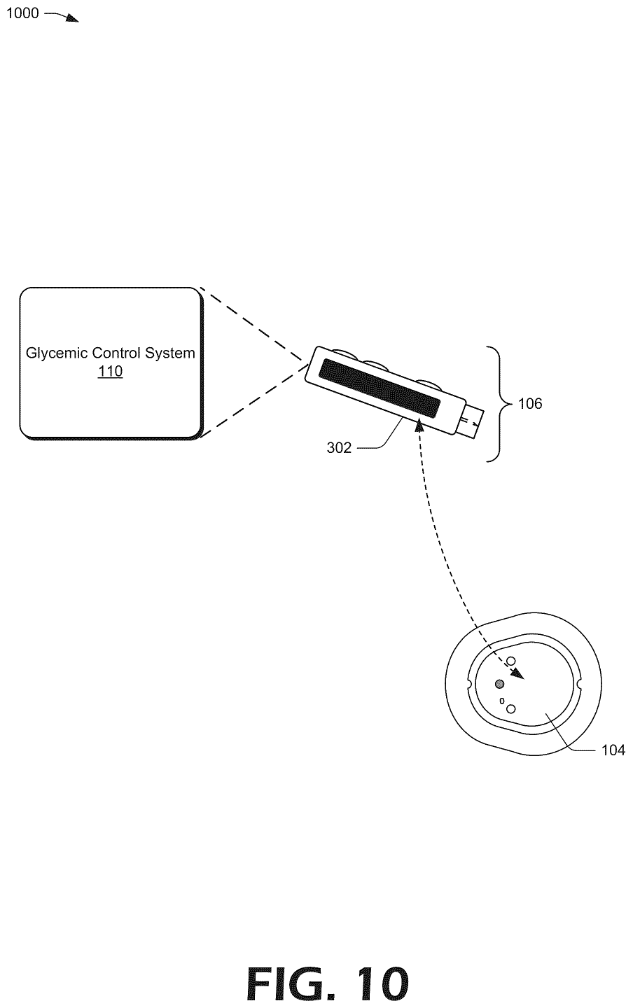

FIG. 10 depicts an example 1000 of a third combination of devices for implementing automatic medicament dosing. The illustrated example 1000 includes the analyte monitoring device 104 and the medicament delivery system 106 having the glycemic control system 110 (depicted as the medicament injection pen 302 in this example).

The illustrated example 1000 does not include the computing device 108, however. This represents a configuration where the analyte monitoring device 104 and computing device 108 are not operably connected the computing device 108. In such configurations, the glycemic control system 110 is included as software, hardware, or a combination thereof on the medicament delivery system 106. For example, the medicament injection pen 302 may be communicatively coupled to the analyte monitoring device 104 (e.g., via the network 114 or another wireless connection) and receive the sensor data 116 (not shown in FIG. 10) directly from the analyte monitoring device 104. The medicament injection pen 302, via the glycemic control system 110, may process the sensor data 116 to generate and execute the instructions 118 (not shown in FIG. 10). Thus, when the analyte monitoring device 104 and the medicament delivery system 106 are configured as in the example 900, those devices can be operably connected to implement glycemic control as an open-loop system or a partially open-loop system.

Having discussed exemplary details of the techniques for automatic medicament delivery, consider now some examples of procedures to illustrate additional aspects of the techniques.

### Example Procedures

This section describes examples of procedures for location-aided glycemic control. Aspects of the procedures may be implemented in hardware, firmware, or software, or a combination thereof. The procedures are shown as a set of blocks that specify operations performed by one or more devices and are not necessarily limited to the orders shown for performing the operations by the respective blocks. In at least some implementations the procedures are performed by a glycemic control system, such as glycemic control system 110.

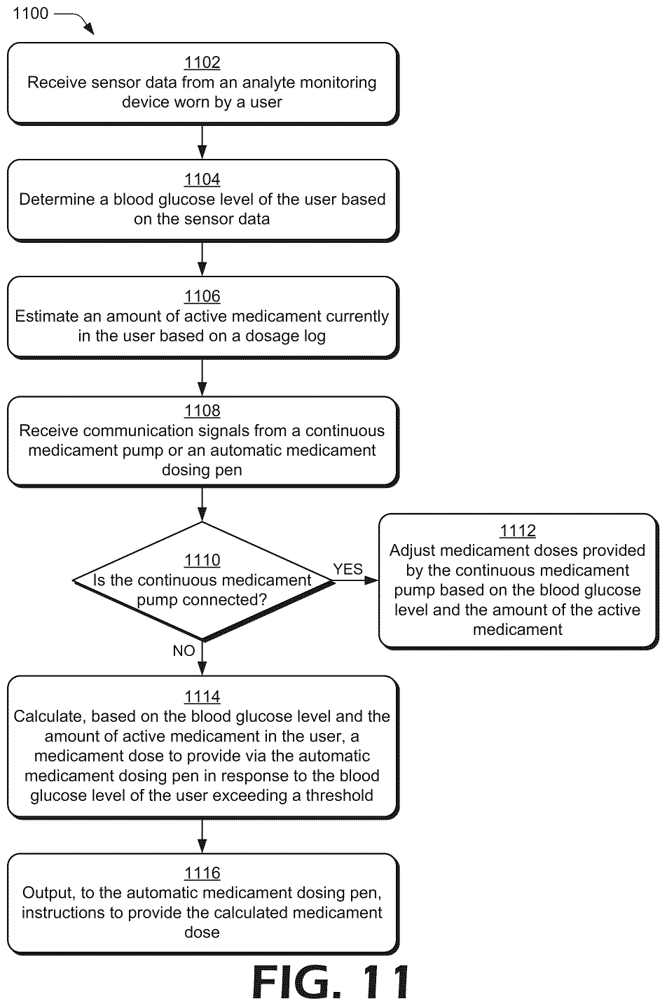

FIG. 11 depicts a procedure 1100 in an example implementation of automatic medicament dosing for glycemic control.

Sensor data is received from an analyte monitoring device worn by a user (block 1102). By way of example, the glycemic control system 110 obtains the sensor data 116 from the analyte monitoring device 104. In accordance with the described techniques, the glycemic control system 110 can receive additional data (e.g., the monitoring data 404) from various sources 402. Examples of the additional monitoring data include proximity data (e.g., between connected devices), analyte data (one or more different analytes or different data about a same analyte from different sources), medicament data, and user data, just to name just a few.

A blood glucose level of the user is determined based on the sensor data (block 1104). By way of example, based on the sensor data 116, the glycemic control system 110 detects an analyte (e.g., glucose) level in the blood or interstitial fluid of the user (e.g., the person 102) and calculates the blood glucose level accordingly.

An amount of active medicament that is currently in the user is estimated based on a dosage log (block 1106). By way of example, the dosage log 408 tracks a time, date, and amount of each medicament delivery, and the medicament tracking engine 120 of the glycemic control system 110 may utilize this information in combination with information regarding a duration of action by the medicament in the user's body (e.g., a duration of insulin action) to estimate (e.g., calculate) the amount of active medicament that is currently in the user's body. In at least one implementation, the medicament tracking engine 120 utilizes the sensor data 116 and/or the historical sensor data 128 to estimate the user's response to previously provided doses of the medicament in calculating the amount of active medicament that is currently in the user. Additionally, or alternatively, the sensor data 116 may include a direct or indirect measurement of the active medicament in the user. In various scenarios, no active medicament is present within the user, such as when all of the previously provided doses of the medicament are expected to be used up and/or no medicament has been provided for at least a threshold duration during which the medicament is expected to be degraded.

Communication signals from a continuous medicament pump or an automatic medicament dosing pen are received (block 1108). By way of example, the glycemic control system 110 (e.g., a device operating the glycemic control system 110, such as the computing device 108) receives an indication of a communicative connection to the continuous medicament pump (e.g., the medicament pump 802) and/or the automatic medicament dosing pen (e.g., the medicament injection pen 302). In at least one implementation, the glycemic control system 110 has one available connection for connecting to the continuous medicament pump or the automatic medicament dosing pen and thus, the automatic medicament dosing pen is not communicatively connected while the continuous medicament pump is connected (and vice versa). For example, the computing device 108 may pair with the analyte monitoring device 104 over a first wireless communication channel (e.g., a first BLE channel) and pair with either the continuous medicament pump or the automatic medicament dosing pen over a second wireless communication channel (e.g., a second BLE channel). Alternatively, the glycemic control system 110 has multiple available connections and may communicatively connect to both of the continuous medicament pump and the automatic medicament dosing pen at the same time.

It is determined if the continuous medicament pump is connected (block 1110). By way of example, the glycemic control system 110 may determine that the continuous medicament pump is connected in response to receiving communication signals from the continuous medicament pump. In contrast, the glycemic control system 110 may determine that the continuous medicament pump is not connected in response to not receiving communication signals from the continuous medicament pump, such as when the continuous medicament pump is powered down, out of range, or otherwise communicatively disconnected. In at least one variation, the glycemic control system 110 may further determine that the continuous medicament pump is not connected in response to the automatic medicament dosing pen being connected, such as when the device operating the glycemic control system 110 is unable to pair with more than one medicament delivery device.

If the continuous medicament pump is connected, medicament doses provided by the continuous medicament pump are adjusted based on the blood glucose level and the amount of the active medicament (block 1112). By way of example, the continuous medicament pump may be operated as a closed-loop system to accurately maintain the user's blood glucose level within a desired blood glucose range. In at least one implementation, the recommendation engine 122 of the glycemic control system 110 generates a recommendation 410 including dosing instructions 416, which may be used by the continuous medicament pump to supply the medicament as a continuous or periodic subcutaneous infusion over time. In this way, excursions from the desired blood glucose range, such as hyperglycemia or hypoglycemia events, may be reduced.

If the continuous medicament pump is not connected, a medicament dose to provide via the automatic medicament dosing pen is calculated based on the blood glucose level and the amount of active medicament in the user in response to the blood glucose level of the user exceeding a threshold (block 1114). By way of example, the threshold may be an upper value of the desired blood glucose range. As one non-limiting example, the threshold is 160 milligrams per deciliter (mg/dL). In at least one implementation, the recommendation engine 122 calculates the medicament dose based on a rate of change in the blood glucose level in addition to or as an alternative to using a single blood glucose value. In at least one implementation, the calculated medicament dose is expected to bring the user's blood glucose level within the desired blood glucose range. It is to be appreciated that an algorithm used by the glycemic control system 110 to calculate the medicament dose is different when the medicament is administered by the automatic medicament dosing pen, and not the continuous medicament pump, due to the discrete injections provided by the automatic medicament dosing pen (in contrast to the continual insertion of cannula of the infusion set 804 of the medicament pump 802 at the insertion site).

Furthermore, in at least one implementation, the calculated medicament dose is divided between multiple injections that are to be administered over a period of time rather than a single injection. The calculated medicament dose may be divided approximately equally between the multiple injections. Alternatively, the calculated medicament dose may be unequally distributed between the multiple injections. For example, a first, initial injection of the multiple injections may include a larger (or smaller) proportion of the calculated medicament dose than a second, subsequent injection of the multiple injections. Furthermore, due to the different timing of the multiple injections compared with the single injection, a cumulative dose of the multiple injections may be different (e.g., larger or smaller) than a comparable dose given via a single injection. In some implementations, after the first injection of the multiple injections is administered, the recommendation engine 122 may adjust an amount and/or timing of one or more of the remaining injections based on sensor data received following the first injection (e.g., received between the first injection and the second injection).

Instructions to provide the calculated medicament dose are output to the automatic medicament dosing pen (block 1116). By way of example, the instructions (e.g., the instructions 118) are communicated to the automatic medicament dosing pen over the wireless connection. The instructions include, for example, the dosing instructions 416 regarding a number of doses, a frequency of the doses, and an amount of medicament in each dose. Thus, the dosing instructions 416 may instruct the automatic medicament dosing pen to administer the calculated medicament dose via one or more injections. In some scenarios, the instructions further include the priming instructions 418, such as when priming of the automatic medicament dosing pen is indicated.

It is to be appreciated that is at least one implementation, the automatic medicament dosing pen includes the glycemic control system 110, and the procedure 1100 may be adapted accordingly. For example, the automatic medicament dosing pen may receive the sensor data, calculate the medicament dose based at least on the sensor data, generate instructions for providing the calculated medicament dose, and execute the instructions for providing the calculated medicament dose without receiving additional instructions from the computing device 108.

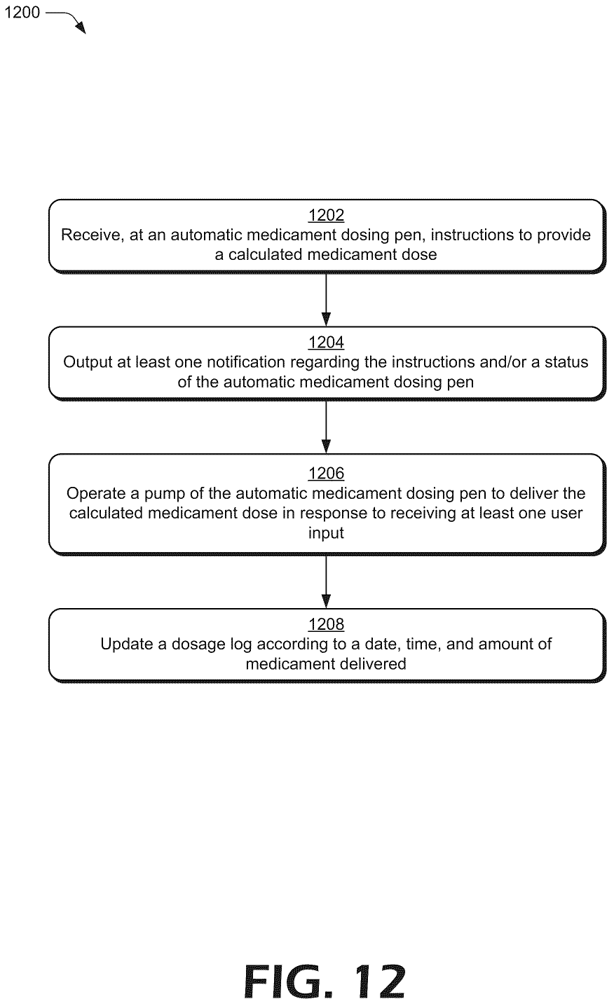

FIG. 12 depicts a procedure 1200 in an example implementation of automatic medicament dosing using an automatic medicament dosing pen (e.g., the medicament injection pen 302).

Instructions to provide a calculated medicament dose are received at the automatic medicament dosing pen (block 1202). By way of example, the instructions (e.g., the instructions 118) include dosing instructions (e.g., the dosing instructions 416) regarding a number of doses, a frequency of the doses, and an amount of medicament in each dose. For example, the calculated medicament dose may be provided via a single injection or several injections given over a pre-defined time period at pre-defined intervals (e.g., as defined by the instructions). Furthermore, in some scenarios, the instructions include priming instructions (e.g., the priming instructions 418), such as when a new needle or medicament cartridge has been inserted into the automatic medicament dosing pen or when air is expected to be present in the needle.

In at least one implementation, the instructions are received from a separate device (e.g., the computing device 108) that includes the glycemic control system 110, such as via a wireless communicative coupling with the separate device. Alternatively, the automatic medicament dosing pen includes the glycemic control system 110 and generates the instructions. Additional details regarding generating the instructions are described herein, for example, at FIG. 4 and FIG. 11.

In at least one implementation, the medicament delivery controls 314 of the medicament injection pen 302 translate the dosing instructions 416 into pump control instructions. The pump control instructions may include, for example, an amount or pulse-width of voltage (or current) to supply to the pump 324 (e.g., from the battery 322) and/or a duration of powering the pump to provide the calculated dose. In an example where the pump 324 includes a stepper motor, the medicament delivery controls 314 may calculate a number of steps to rotate the stepper motor based on the calculated medicament dose and further determine operation parameters that will cause the stepper motor to rotate the calculated number of steps.

At least one notification regarding the instructions and/or a status of the automatic medicament dosing pen is output (block 1204). By way of example, the at least one notification may be output via one or more user interfaces, such as a user interface of the computing device 108 and/or the user interface 306. The at least one notification may be, for example, a visual notification (such as a text-based message, flashing lights, etc.), an audible sound or alert, and/or a tactile notification (e.g., a vibration or other type of haptic feedback). Non-limiting examples of the at least one notification include a first notification indicating the calculated medicament dose, a second notification instructing the user to initiate priming, a third notification indicating the priming is in progress, a fourth notification indicating the priming is complete, a fifth notification instructing the user to initiate administration of the medicament, a sixth notification indicating dosing is in progress, and a seventh notification indicating dosing is complete. Any, all, or various combinations of these non-limiting examples as well as others not specifically listed may be output at various times (e.g., in a sequence) after receiving the instructions and/or in response to receiving user input, examples of which are described herein.

A pump of the automatic medicament dosing pen is operated to deliver the calculated medicament dose in response to receiving at least one user input (block 1206). By way of example, operating the pump to deliver the calculated medicament dose includes supplying electrical power from the battery 322 to the electric motor of the pump according to the pump control instructions in order to dispense the calculated medicament dose from the medicament reservoir 316, through the needle 304, and into the user. In one or more implementations, the automatic medicament dosing pen may receive user input via the user interface 306 and/or at least one button 308. Additionally, or alternatively, the automatic medicament dosing pen may receive user input via the communicatively coupled but separate device (e.g., the computing device 108). The at least one user input may include an indication that the needle 304 is inserted in the user.

Additional or alternative non-limiting examples of the at least one user input include a verification input verifying the calculated medicament dose (e.g., in response to the first notification), a priming input configured to initiate priming of the automatic medicament dosing pen (e.g., in response to the second notification), and an injection input to initiate pumping of the medicament once the needle 304 of the automatic medicament dosing pen has been inserted into the skin (e.g., in response to the fifth notification). Any, all, or various combinations of these non-limiting examples as well as others not specifically listed may be received in order to operate the pump 324 to deliver the calculated medicament dose. Furthermore, it is to be understood that in the context of automatic medicament dosing, the at least one user input does not include inputs to manually set the medicament dose to be delivered.

In an example scenario, the automatic medicament dosing pen outputs (e.g., via the user interface 306) the first notification indicating the calculated medicament dose and receives the verification input by the user depressing a first button of the at least one button 308. In response to receiving the verification input, the automatic medicament dosing pen outputs the second notification instructing the user to initiate priming. The automatic medicament dosing pen receives the priming input by the user depressing a second button of the at least one button 308, and in response to receiving the priming input, the automatic medicament dosing pen primes the needle 304 by operating the pump 324 to deliver a priming amount. While operating the pump 324 to deliver the priming amount, the automatic medicament dosing pen outputs the third notification indicating the priming is in progress. In response to completion of the priming, the automatic medicament dosing pen outputs the fourth notification indicating the priming is complete, followed by the fifth notification instructing the user to initiate administration of the medicament. The automatic medicament dosing pen receives the injection input by the user depressing a third button of the at least one button 308 and operates the pump 324 to deliver the calculated medicament dose in response thereto. While operating the pump 324 to deliver the calculated medicament dose, the automatic medicament dosing pen outputs the sixth notification indicating dosing is in progress. In response to the calculated medicament dose being delivered (e.g., the pump 324 is no longer actively delivering the medicament), the automatic medicament dosing pen outputs the seventh notification indicating dosing is complete.

In another example scenario, the automatic medicament dosing pen outputs (e.g., via the user interface 306) the first notification indicating the calculated medicament dose and receives the verification input by the user depressing the at least one button 308 a first time. In response to receiving the verification input, the automatic medicament dosing pen outputs the fifth notification instructing the user to initiate administration of the medicament. The automatic medicament dosing pen receives the injection input by the user depressing the at least one button 308 a second time and operates the pump 324 to deliver the calculated medicament dose in response thereto. While operating the pump 324 to deliver the calculated medicament dose, the automatic medicament dosing pen outputs the sixth notification indicating dosing is in progress. In response to the calculated medicament dose being delivered (e.g., the pump 324 is no longer actively delivering the medicament), the automatic medicament dosing pen no longer outputs the sixth notification.

In yet another example scenario, the automatic medicament dosing pen outputs (e.g., via the user interface 306) the first notification indicating the calculated medicament dose and receives the injection input, which functions to both verify the calculated medicament dose and initiate the injection. In response to receiving the injection input, the automatic medicament dosing pen operates the pump 324 to deliver the calculated medicament dose. Upon completing delivery of the calculated medicament dose, the automatic medicament dosing pen no longer outputs the first notification, which indicates that the calculated medicament dose has been delivered.

In at least one implementation, the injection input includes a prolonged button press or multiple button presses in order to confirm that the input is intentional. For example, the injection input may include the button being depressed for at least a threshold duration (e.g., 2 seconds). In this way, inadvertent medicament pumping may be avoided.

A dosage log is updated according to a date, time, and amount of medicament delivered (block 1208). By way of example, the dosage log 408 may be updated in order to track an amount of active medicament in the user (e.g., “insulin on board”) as well as a remaining amount of the medicament in the medicament reservoir 316.

It is to be appreciated that at least portions of block 1204 through block 1208 may be repeated to perform each injection when the calculated medicament dose is administered via more than one injection. For example, the automatic medicament dosing pen may output additional notifications and receive additional user input after a pre-determined duration elapses after a first portion of the calculated medicament dose is administered in order to administer a second portion of the calculated medicament dose, and so forth.

Having described examples of procedures in accordance with one or more implementations, consider now an example of a system and device that can be utilized to implement the various techniques described herein.

### Example System and Device

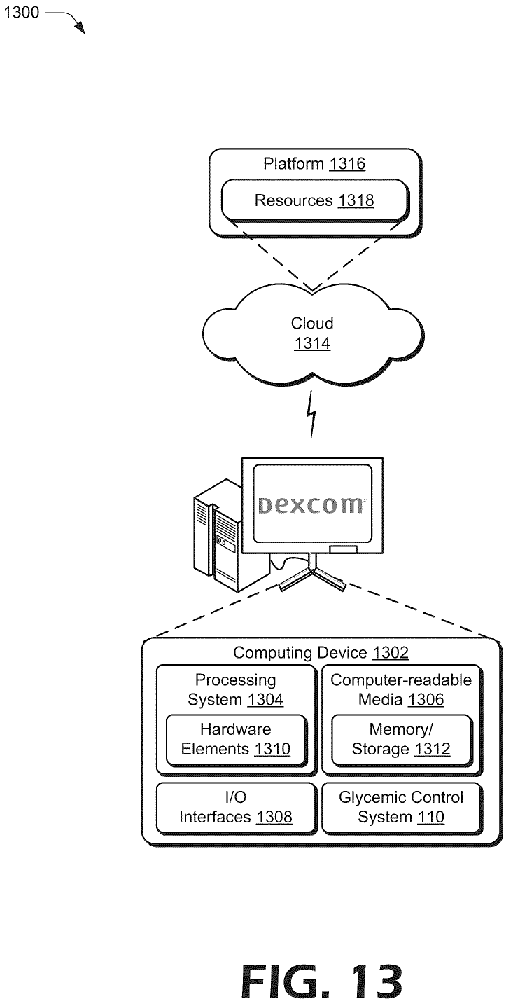

FIG. 13 illustrates an example of a system generally at 1300 that includes an example of a computing device 1302 that is representative of one or more computing systems and/or devices that may implement the various techniques described herein. This is illustrated through inclusion of the glycemic control system 110. The computing device 1302 may be, for example, a server of a service provider, a device associated with a client (e.g., a client device), an on-chip system, and/or any other suitable computing device or computing system.

The example computing device 1302 as illustrated includes a processing system 1304, one or more computer-readable media 1306, and one or more I/O interfaces 1308 that are communicably coupled, one to another. Although not shown, the computing device 1302 may further include a system bus or other data and command transfer system that couples the various components, one to another. A system bus can include any one or combination of different bus structures, such as a memory bus or memory controller, a peripheral bus, a universal serial bus, and/or a processor or local bus that utilizes any of a variety of bus architectures. A variety of other examples are also contemplated, such as control and data lines.

The processing system 1304 is representative of functionality to perform one or more operations using hardware. Accordingly, the processing system 1304 is illustrated as including hardware elements 1310 that may be configured as processors, functional blocks, and so forth. This may include implementation in hardware as an application specific integrated circuit or other logic device formed using one or more semiconductors. The hardware elements 1310 are not limited by the materials from which they are formed or the processing mechanisms employed therein. For example, processors may be comprised of semiconductor(s) and/or transistors (e.g., electronic integrated circuits (ICs)). In such a context, processor-executable instructions may be electronically-executable instructions.

The computer-readable media 1306 is illustrated as including memory/storage 1312. The memory/storage 1312 represents memory/storage capacity associated with one or more computer-readable media. The memory/storage 1312 may include volatile media (such as random access memory (RAM)) and/or nonvolatile media (such as read only memory (ROM), Flash memory, optical disks, magnetic disks, and so forth). The memory/storage 1312 may include fixed media (e.g., RAM, ROM, a fixed hard drive, and so on) as well as removable media (e.g., Flash memory, a removable hard drive, an optical disc, and so forth). The computer-readable media 1306 may be configured in a variety of other ways as further described below.

Input/output interface(s) 1308 are representative of functionality to allow a user to enter commands and information to computing device 1302, and also allow information to be presented to the user and/or other components or devices using various input/output devices. Examples of input devices include a keyboard, a cursor control device (e.g., a mouse), a microphone, a scanner, touch functionality (e.g., capacitive or other sensors that are configured to detect physical touch), a camera (e.g., which may employ visible or non-visible wavelengths such as infrared frequencies to recognize movement as gestures that do not involve touch), and so forth. Examples of output devices include a display device (e.g., a monitor or projector), speakers, a printer, a network card, tactile-response device, and so forth. Thus, the computing device 1302 may be configured in a variety of ways as further described below to support user interaction.

Various techniques may be described herein in the general context of software, hardware elements, or program modules. Generally, such modules include routines, programs, objects, elements, components, data structures, and so forth that perform particular tasks or implement particular abstract data types. The terms “module,” “functionality,” and “component” as used herein generally represent software, firmware, hardware, or a combination thereof. The features of the techniques described herein are platform-independent, meaning that the techniques may be implemented on a variety of commercial computing platforms having a variety of processors.

An implementation of the described modules and techniques may be stored on or transmitted across some form of computer-readable media. The computer-readable media may include a variety of media that may be accessed by the computing device 1302. By way of example, and not limitation, computer-readable media may include “computer-readable storage media” and “computer-readable signal media.”

“Computer-readable storage media” may refer to media and/or devices that enable persistent and/or non-transitory storage of information in contrast to mere signal transmission, carrier waves, or signals per se. Thus, computer-readable storage media refers to non-signal bearing media. The computer-readable storage media includes hardware such as volatile and non-volatile, removable and non-removable media and/or storage devices implemented in a method or technology suitable for storage of information such as computer readable instructions, data structures, program modules, logic elements/circuits, or other data. Examples of computer-readable storage media may include, but are not limited to, RAM, ROM, EEPROM, flash memory or other memory technology, CD-ROM, digital versatile disks (DVD) or other optical storage, hard disks, magnetic cassettes, magnetic tape, magnetic disk storage or other magnetic storage devices, or other storage device, tangible media, or article of manufacture suitable to store the desired information and which may be accessed by a computer.

“Computer-readable signal media” may refer to a signal-bearing medium that is configured to transmit instructions to the hardware of the computing device 1302, such as via a network. Signal media typically may embody computer readable instructions, data structures, program modules, or other data in a modulated data signal, such as carrier waves, data signals, or other transport mechanism. Signal media also include any information delivery media. The term “modulated data signal” means a signal that has one or more of its characteristics set or changed in such a manner as to encode information in the signal. By way of example, and not limitation, communication media include wired media such as a wired network or direct-wired connection, and wireless media such as acoustic, RF, infrared, and other wireless media.

As previously described, hardware elements 1310 and computer-readable media 1306 are representative of modules, programmable device logic and/or fixed device logic implemented in a hardware form that may be employed in some embodiments to implement at least some aspects of the techniques described herein, such as to perform one or more instructions. Hardware may include components of an integrated circuit or on-chip system, an application-specific integrated circuit (ASIC), a field-programmable gate array (FPGA), a complex programmable logic device (CPLD), and other implementations in silicon or other hardware. In this context, hardware may operate as a processing device that performs program tasks defined by instructions and/or logic embodied by the hardware as well as a hardware utilized to store instructions for execution, e.g., the computer-readable storage media described previously.

Combinations of the foregoing may also be employed to implement various techniques described herein. Accordingly, software, hardware, or executable modules may be implemented as one or more instructions and/or logic embodied on some form of computer-readable storage media and/or by one or more hardware elements 1310. The computing device 1302 may be configured to implement particular instructions and/or functions corresponding to the software and/or hardware modules. Accordingly, implementation of a module that is executable by the computing device 1302 as software may be achieved at least partially in hardware, e.g., through use of computer-readable storage media and/or hardware elements 1310 of the processing system 1304. The instructions and/or functions may be executable/operable by one or more articles of manufacture (for example, one or more computing devices 1302 and/or processing systems 1304) to implement techniques, modules, and examples described herein.

The techniques described herein may be supported by various configurations of the computing device 1302 and are not limited to the specific examples of the techniques described herein. This functionality may also be implemented all or in part through use of a distributed system, such as over a “cloud” 1314 via a platform 1316 as described below.

The cloud 1314 includes and/or is representative of a platform 1316 for resources 1318. The platform 1316 abstracts underlying functionality of hardware (e.g., servers) and software resources of the cloud 1314. The resources 1318 may include applications and/or data that can be utilized while computer processing is executed on servers that are remote from the computing device 1302. Resources 1318 can also include services provided over the Internet and/or through a subscriber network, such as a cellular or Wi-Fi network.

The platform 1316 may abstract resources and functions to connect the computing device 1302 with other computing devices. The platform 1316 may also serve to abstract scaling of resources to provide a corresponding level of scale to encountered demand for the resources 1318 that are implemented via the platform 1316. Accordingly, in an interconnected device embodiment, implementation of functionality described herein may be distributed throughout the system 1300. For example, the functionality may be implemented in part on the computing device 1302 as well as via the platform 1316 that abstracts the functionality of the cloud 1314.

### CONCLUSION

Although the systems and techniques have been described in language specific to structural features and/or methodological acts, it is to be understood that the systems and techniques defined in the appended claims are not necessarily limited to the specific features or acts described. Rather, the specific features and acts are disclosed as example forms of implementing the claimed subject matter.

What is claimed is:

1. A method comprising:
calculating a dose of a medicament to administer to a user via a medicament injection pen based on analyte monitoring data;
receiving a user input to initiate an injection of the medicament; and
operating the medicament injection pen to administer the calculated dose to the user in response to receiving the user input to initiate the injection.

2. The method of claim 1, wherein the operating the medicament injection pen to administer the calculated dose of the medicament to the user in response to receiving the user input to initiate the injection comprises powering a motorized pump of the medicament injection pen to flow the calculated dose from a medicament reservoir of the medicament injection pen through a needle of the medicament injection pen.

3. The method of claim 1, further comprising estimating an amount of active medicament present in the user based on a dosage log of previously injected doses of the medicament, and wherein the calculating the dose of the medicament to administer to the user via the medicament injection pen is further based on the amount of active medicament present in the user.

4. The method of claim 1, further comprising determining a blood glucose level of the user based on the analyte monitoring data, the analyte monitoring data received from an analyte monitoring device worn by the user, and wherein the calculating the dose of the medicament to administer to the user via the medicament injection pen based on the analyte monitoring data is in response to the blood glucose level of the user being greater than a threshold.

5. The method of claim 1, further comprising outputting a notification regarding the calculated dose prior to receiving the user input to initiate the injection.

6. The method of claim 5, wherein the outputting the notification comprises outputting the notification via a user interface of the medicament injection pen, and wherein the receiving the user input to initiate the injection comprises receiving the user input via the user interface or an electronic button of the medicament injection pen.

7. The method of claim 5, wherein outputting the notification comprises outputting the notification via a user interface of a computing device that is communicatively coupled to the medicament injection pen.

8. The method of claim 5, wherein the notification comprises a first notification indicating the calculated dose of the medicament, and wherein the receiving the user input to initiate the injection comprises receiving a first user input verifying the calculated dose of the medicament after outputting the first notification.

9. The method of claim 8, further comprising outputting, in response to receiving the first user input, a second notification including instructions to position the medicament injection pen to inject the calculated dose of the medicament into the user, and wherein the receiving the user input to initiate the injection further comprises receiving a second user input confirming positioning of the medicament injection pen after outputting the second notification.

10. The method of claim 1, wherein the medicament injection pen is communicatively coupled to a computing device, and the operating the medicament injection pen to administer the calculated dose to the user is further in response to the medicament injection pen receiving, from the computing device, instructions to administer the calculated dose.

11. The method of claim 10, wherein the receiving the user input to initiate the injection of the medicament comprises receiving the user input via a user interface of the computing device.

12. The method of claim 10, wherein the calculating the dose of the medicament to administer to the user via the medicament injection pen based on the analyte monitoring data is performed by the computing device in response to a communicative coupling between the computing device and a continuous medicament delivery pump being disconnected.

13. A system comprising:
an analyte monitoring device configured to obtain analyte measurements of a user;
a medicament injection pen to administer a medicament to the user; and
at least a memory and a processor to perform operations comprising:
calculating, based on the analyte measurements, a blood glucose level of the user;
calculating an amount of the medicament to administer to the user in response to the blood glucose level of the user exceeding a threshold; and
providing instructions causing the medicament injection pen to deliver the amount of the medicament to the user in response to receiving an indication that a needle of the medicament injection pen is inserted into the user.

14. The system of claim 13, wherein the amount of the medicament is determined based at least in part on the analyte measurements of the user obtained from the analyte monitoring device.

15. The system of claim 13, wherein the instructions cause the medicament injection pen to deliver the amount of the medicament to the user without receiving user input setting the amount.

16. The system of claim 13, wherein the medicament comprises insulin, and wherein the medicament injection pen comprises a motorized pump.

17. The system of claim 13, wherein the medicament injection pen has a user interface for receiving user input causing the medicament injection pen to initiate delivery of the amount of the medicament to the user.

18. The system of claim 13, wherein the medicament injection pen includes a user interface for establishing wireless communication with an external device.

19. The system of claim 18, wherein the external devices is the analyte monitoring system.

20. The system of claim 18, wherein the external device includes a computing device in which the at least memory and the processor are located.

21. The system of claim 13, wherein the medicament injection pen includes a user interface that selectively activates a retraction mechanism to extend and retract the needle from a cap.

22. The system of claim 13, wherein the memory and the processor are included in the medicament injection pen.

23. The system of claim 13, further comprising a computing device in which the memory and the processor are located, the medicament injection pen including a communication module for receiving the instructions from the computing device.

24. The system of claim 13, wherein the computing device receives the analyte measurements from the analyte monitoring device over a wireless connection.

25. The system of claim 16, wherein the motorized pump includes a stepper motor in which each rotation is divided into a pre-defined number of steps.

26. The system of claim 25, wherein each of the steps of the stepper motor causes a known quantity of the medicament to be delivered via the needle.

27. The system of claim 26, wherein the memory and the processor calculate the number of steps to rotate the stepper motor to extract the amount of medicament to be delivered via the needle.

28. The system of claim 13, wherein the medicament injection pen includes a medicament reservoir in which the medicament is housed, wherein the medicament injection pen is configured to identify a type of medicament in the medicament reservoir based on an indicator in the medicament reservoir.

29. The system of claim 13, wherein the medicament injection pen includes a display for displaying analyte measurements.

30. The system of claim 13, wherein the medicament injection pen includes a medicament sensor for measuring a systemic level of the medicament in the user.

31. The system of claim 30, wherein the medicament injection pen includes a safety module for preventing medicament delivery based on medicament measurements from the medicament sensor.

32. The system of claim 13, wherein the amount of the medicament to administer is a bolus dose and calculating the amount includes dividing the bolus dose into a plurality of smaller doses that are to be administered at intervals determined by the memory and the processor.

33. The system of claim 32, wherein determining to divide the bolus does into a plurality of smaller doses is based on receipt of user input.

34. A method comprising:
calculating a bolus amount of medicament to administer to a user via a medicament injection pen in response to a blood glucose level of the user exceeding a threshold;
receiving an indication that a needle of the medicament injection pen is inserted into the user; and
operating a motorized pump of the medicament injection pen to deliver the bolus amount of the medicament to the user via at least one injection in response to receiving the indication that the needle of the medicament injection pen is inserted into the user.

35. The method of claim 34 further comprising obtaining glucose measurements of the user from a glucose monitoring device worn by the user, and wherein the calculating the bolus amount of the medicament to administer to the user in response to the blood glucose level of the user exceeding a threshold comprises calculating the bolus amount based at least in part on the glucose measurements.

36. The method of claim 35, further comprising determining an active amount of the medicament in the user based at least in part on a dosage log of medicament delivery to the user, and wherein the calculating the bolus amount of the medicament to administer to the user in response to the blood glucose level of the user exceeding a threshold comprises calculating the bolus amount further based on the active amount of the medicament.

37. The method of claim 34, further comprising outputting at least one notification regarding administering the calculated bolus amount of the medicament to the user via a user interface, and wherein the receiving the indication that the needle of the medicament injection pen is inserted into the user comprises receiving the indication via the user interface after outputting the at least one notification.

38. The system of claim 34, wherein the bolus amount of the medicament is determined based at least in part on the analyte measurements of the user obtained from an analyte monitoring device.

39. The system of claim 34, wherein the bolus amount of the medicament is delivered to the user without receiving user input setting the amount.

40. The method of claim 37, wherein the medicament injection pen has a user interface for receiving user input causing the motorized pump to initiate delivery of the amount of the medicament to the user.

41. The method of claim 37, further comprising establishing wireless communication between the medicament injection pen and an external device.

42. The method of claim 41, wherein the external devices is a glucose monitoring system that is configured to obtain the blood glucose level of the user.

43. The method of claim 41, wherein the external device includes a computing device the computing device performing the calculating and communicating the calculated bolus amount to the medicament injection pen.

44. The method of claim 37, further comprising receiving user input that selectively activates a retraction mechanism in the medicament injection pen to extend and retract the needle from a cap.

45. The method of claim 37, wherein the calculating is performed by the medicament injection pen.

46. The method of claim 37, wherein the motorized pump includes a stepper motor in which each rotation is divided into a pre-defined number of steps.

47. The method of claim 46, wherein each of the steps of the stepper motor causes a known quantity of the medicament to be delivered via the needle.

48. The method of claim 47, further comprising calculating the number of steps to rotate the stepper motor to extract the amount of medicament to be delivered via the needle.

49. The method of claim 37, further comprising identifying a type of medicament in a medicament reservoir of the medicament injection pen based on an indicator in the medicament reservoir.

50. The method of claim 37, further comprising receiving medicament measurements data from a medicament sensor, the medicament measurements indicating a systemic level of the medicament in the user.

51. The method of claim 20, further comprising preventing medicament delivery based on the medicament measurements from the medicament sensor.

52. The method of claim 37, wherein calculating the bolus amount includes dividing the bolus amount into a plurality of smaller doses that are to be administered at intervals determined by the memory and the processor.

53. The method of claim 52, further comprising receiving user input that causes the bolus amount to be divided into the plurality of smaller doses.
